(19) United States
(12) Patent Application Publication
(10) Pub. No.: US 2016/0068904 A1
(43) Pub. Date: Mar. 10, 2016

(54) METHODS OF SKIN ANALYSIS AND USES THEREOF

(71) Applicant: SKINSHIFT, Austin, TX (US)

(72) Inventor: Ruthie Harper, Austin, TX (US)

(73) Assignee: Skinshift, Austin, TX (US)

(21) Appl. No.: 14/786,673

(22) PCT Filed: Apr. 24, 2014
(86) PCT No.: PCT/US14/35298

§ 371 Date: Oct. 23, 2015

Related U.S. Application Data
(60) Provisional application No. 61/815,604, filed on Apr. 24, 2013.

(51) Int. Cl. C12Q 1/68; G06F 19/18

(52) U.S. Cl. CPC C12Q 1/6876; G06F 19/18; C12Q 2600/156; C12Q 2600/148

(57) ABSTRACT

Disclosed herein are skin analysis methods and assays which objectively identify biological strengths and weaknesses in a patient's genetic coding that affect the health and beauty of their skin. Generated results can be utilized to develop personalized skin care and nutritional regimens to prevent, reduce and treat skin deterioration, disorders and diseases. In some embodiments, a method of characterizing a subject's skin is provided which includes generating a personalized skin profile by determining a subject's genetic potential in at least one area of skin health by analyzing one or more skin health-associated single nucleotide polymorphisms or other genetic marker associated with the particular area of skin health being assessed in a sample obtained from the subject. The generated skin profile reveals the subject's genetic strengths, weaknesses and/or risks related to the one or more areas of skin health allowing a personalized skincare and/or nutritional regimen to be developed and implemented.

### CROSS REFERENCE TO RELATED APPLICATION

This application claims the benefit under 35 U.S.C. 119(e) of the earlier filing date of U.S. Patent Application No. 61/815,604 filed on Apr. 24, 2013, which is incorporated herein by reference in its entirety.

### FIELD

This disclosure relates to the field of skincare, more specifically to skin analysis methods and assays for determining and improving the appearance of the skin and methods and protocols for treatment based on these methods or assays.

### BACKGROUND

Many methods are currently used to improve skin quality, health or appearance, such as methods to reduce wrinkles, reduce acne, inflammation or irritation, reduce brown or red spots, soften fine lines, wrinkles or scars, protect against the sun and other environmental factors and protect against internal factors that damage and age the skin such as glycation and inflammation. Currently, skin care products used to improve skin quality and health are administered based upon the physically displayed symptoms of the individual and are not focused on determining the underlying causes of such conditions or causes of the change in appearance. Without considering underlying or causative factors involved in skin quality, health and appearance, the treatment often is not effective, especially for preventing the skincare condition. Specifically, the disorder, disease or change in appearance has already developed when treatment is implemented if treatment is not commenced until physical symptoms are displayed. A need remains for methods to determine the health of a subject's skin and propensity toward deleterious skin changes and degeneration by identifying factors, including genetic factors, which contribute to skin quality so that steps can be taken to prevent, reduce or inhibit skincare conditions, disorders or diseases.

### SUMMARY

Disclosed herein are skin analysis methods and assays which objectively identify biological strengths and weaknesses in a patient's genetic coding that affect the health and beauty of their skin. The generated results can then be utilized to develop a personalized skin care and nutritional regimen or protocol to prevent, reduce and treat skin deterioration, disorders and diseases. In some embodiments, a method of characterizing a subject's skin is provided which includes generating a personalized skin profile. In some examples, generating a personalized skin profile includes determining a subject's genetic potential in at least one area of skin health by analyzing one or more skin health-associated single nucleotide polymorphisms (SNPs) or other genetic markers associated with the particular area of skin health being assessed in a sample obtained from the subject. In some examples, a subject's genetic potential is determined in one, two, three, four, five or more areas of skin health by analyzing one or more skin health-associated SNPs or other genetic markers associated with the five areas of skin health in a sample obtained from the subject. The one or more areas of skin health can include areas such as collagen formation, sun protection, antioxidant protection, glycation protection and inflammation control. The generated skin profile reveals the subject's genetic strengths, weaknesses and/or risks related to the one or more areas of skin health thereby allowing a personalized skincare and/or nutritional regimen to be developed and implemented.

The foregoing and other features and advantages of the disclosure will become more apparent from the following detailed description of several embodiments, which proceeds with reference to the accompanying figures.

### BRIEF DESCRIPTION OF THE FIGURES

FIGS. 1A-1C validate SNP: MMP-1/Collagen Formation 11q21q22 for use in disclosed methods and protocols.

### DETAILED DESCRIPTION OF SEVERAL EMBODIMENTS

### I. Introduction

The Human Genome Project was one of the greatest accomplishments in scientific history as it laid out the entire base code for the human genome. Following its completion in 2003, scientists began finding human population variations in the base code and began probing to understand what these variations meant. Issues raised have included what encompasses these variations, whether they are mutations, and what, if anything is there biological significance.

Certain variations were found with consistent frequencies, indicating that they were not simple mutations but rather had biological significance in human function. This was supported when scientists determined that these variations had been encoded into the genetic messaging and had been passed down through generations obtaining certain frequencies in the DNA coding.

These variations were found to be single nucleotide polymorphisms (SNPs) or differences in the base code pattern in one person's DNA compared to that of another. Scientists began an exhaustive research project to create a data bank of SNP's, determining where SNP's occur in the DNA code, with what frequency, and what they coded for, if anything, in contributing to differences in human body functions. “Active” SNP's were found to be located in coding portions of the DNA that changed biological function, giving credence to the conclusion that active SNPs serve a purpose in the body, unlike random and functionally irrelevant mutations.

Further research found that certain SNP's occurred and survived in human DNA coding for Darwinian-type reasons—they supported both function and survival in certain populations. This answered the question as to why they became encoded in DNA and passed down from one generation to the next. For example, populations that moved to Northern climates with less sun developed SNP's for lighter skin to allow more Vitamin D to be produced in the skin. The higher levels of Vitamin D protected women against rickets, which was associated with higher mortality rates in childbirth as a result of the disease causing softening and deformity of pelvic bones. Higher levels of Vitamin D ensured better survival for both the child and the mother leading to a preference for this DNA coding.

Herein is disclosed personalized skincare based upon the inventor identifying SNPs or other genetic markers pertinent to the function, health and beauty of the skin and utilizing such SNPs or other genetic markers to determine a subject's skin genetic potential for one or more of the following five key categories of skin health and function:
Collagen Formation Factor—ability of the skin to generate and maintain healthy and ideal collagen;
Sun Protection Factor—ability of the skin to protect against the sun and other environmental insults;
Antioxidant Protection Factor—functioning level of internal antioxidant systems in a subject to keep your skin young and healthy;
Inflammation Control Factor—ability to manage inflammation affecting a subject's skin health and beauty;
Glycation Protection Factor—ability a subject's body to process sugar which when not managed well can affect the health and beauty of the skin.

The generated skin profile reveals the subject's genetic strengths, weaknesses and/or risks related to the one or more areas of skin health thereby allowing one or more of the disclosed skincare and/or nutritional protocols/treatments to be developed and provided.

Although the methods and assays described below primarily concern use of SNPs as the genetic marker it is contemplated that any genetic marker such as RFLP (or Restriction fragment length polymorphism), SSLP (or Simple sequence length polymorphism), AFLP (or Amplified fragment length polymorphism), RAPD (or Random amplification of polymorphic DNA), VNTR (or Variable number tandem repeat), SSR Microsatellite polymorphism, (or Simple sequence repeat), STR (or Short tandem repeat), SFP (or Single feature polymorphism), DArT (or Diversity Arrays Technology), and RAD markers (or Restriction site associated DNA markers) may be used in such methods and assays.

### II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). As used herein and in the appended claims, the singular forms “a”, “an”, and “the” include plural referents unless the context clearly dictates otherwise. Unless defined otherwise, all technical and scientific terms used herein have the same meaning as commonly understood to one of ordinary skill in the art to which this disclosure belongs. “Comprising” means “including.” All publications, patent applications, patents, and other references mentioned herein are incorporated by reference in their entirety.

DNA (deoxyribonucleic acid): DNA is a long chain polymer which comprises the genetic material of most living organisms (some viruses have genes comprising ribonucleic acid (RNA)). The repeating units in DNA polymers are four different nucleotides, each of which comprises one of the four bases, adenine, guanine, cytosine and thymine bound to a deoxyribose sugar to which a phosphate group is attached. Triplets of nucleotides (referred to as codons) code for each amino acid in a polypeptide, or for a stop signal (termination codon). The term codon is also used for the corresponding (and complementary) sequences of three nucleotides in the mRNA into which the DNA sequence is transcribed.

Unless otherwise specified, any reference to a DNA molecule is intended to include the reverse complement of that DNA molecule. Except where single-strandedness is required by the text herein, DNA molecules, though written to depict only a single strand, encompass both strands of a double-stranded DNA molecule. Thus, a reference to the nucleic acid molecule that encodes a protein, or a fragment thereof, encompasses both the sense strand and its reverse complement. Thus, for instance, it is appropriate to generate probes or primers from the reverse complement sequence of the disclosed nucleic acid molecules.

Gene: A segment of DNA that contains the coding sequence for a protein, wherein the segment may include promoters, exons, introns, and other untranslated regions that control expression.

Genetic marker: A gene or DNA sequence with a known location on a chromosome that can be used to identify individuals or species. It can be described as a variation (which may arise due to mutation or alteration in the genomic loci) that can be observed. A genetic marker may be a short DNA sequence, such as a sequence surrounding a single base-pair change (single nucleotide polymorphism, SNP), or a long one, like minisatellites. Some commonly used types of genetic markers are RFLP (or Restriction fragment length polymorphism), SSLP (or Simple sequence length polymorphism), AFLP (or Amplified fragment length polymorphism), RAPD (or Random amplification of polymorphic DNA), VNTR (or Variable number tandem repeat), SSR Microsatellite polymorphism, (or Simple sequence repeat), SNP (or Single nucleotide polymorphism), STR (or Short tandem repeat), SFP (or Single feature polymorphism), DArT (or Diversity Arrays Technology), and RAD markers (or Restriction site associated DNA markers). Molecular genetic markers can be divided into two classes a) biochemical markers which detect variation at the gene product level such as changes in proteins and amino acids and b) molecular markers which detect variation at the DNA level such as nucleotide changes: deletion, duplication, inversion and/or insertion. Disclosed herein are methods and assays for using genetic markers, such as, but not limited to, single nucleotide polymorphisms (SNPs) associated skin health to generate a personalized skin profile.

Genetic predisposition: Susceptibility of a subject to a particular condition or disease, such as a skin care condition or related disease. Detecting a genetic predisposition can include, but does not necessarily include, detecting the presence of the condition or disease itself, such as but not limited to an early stage of the condition or disease process. Detecting a genetic predisposition also includes detecting the risk of developing the condition or disease, and determining the susceptibility of that subject to developing the condition or disease or to having a poor prognosis for the disease. Thus, if a subject has a genetic predisposition to a skin care disorder or disease they do not necessarily develop the disorder or disease but are at risk for developing the disorder or disease.

Genomic target sequence: A sequence of nucleotides located in a particular region in the human genome that corresponds to one or more specific genetic abnormalities, such as a nucleotide polymorphism, a deletion, an insertion, or an amplification. The target can be for instance a coding sequence; it can also be the non-coding strand that corresponds to a coding sequence. The target can also be a non-coding sequence, such as an intronic sequence.

Isolated: An “isolated” biological component (such as a nucleic acid molecule, protein or organelle) has been substantially separated or purified away from other biological components in the cell of the organism in which the component naturally occurs, i.e., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Locus: A location on a chromosome or DNA molecule corresponding to a gene or a physical or phenotypic feature, where physical features include polymorphic sites.

Polymorphism: A variation in a gene sequence. The polymorphisms can be those variations (DNA sequence differences) which are generally found between individuals or different ethnic groups and geographic locations which, while having a different sequence, produce functionally equivalent gene products. Typically, the term can also refer to variants in the sequence which can lead to gene products that are not functionally equivalent. Polymorphisms also encompass variations which can be classified as alleles and/or mutations which can produce gene products which may have an altered function. Polymorphisms also encompass variations which can be classified as alleles and/or mutations which either produce no gene product or an inactive gene product or an active gene product produced at an abnormal rate or in an inappropriate tissue or in response to an inappropriate stimulus. Alleles are the alternate forms that occur at the polymorphism.

Polymorphisms can be referred to, for instance, by the nucleotide position at which the variation exists, by the change in amino acid sequence caused by the nucleotide variation, or by a change in some other characteristic of the nucleic acid molecule or protein that is linked to the variation.

In the instant application “polymorphism” refers a traditional definition, in that the definition “polymorphism” means that the minor allele frequency must be greater than at least 1%.

A “single nucleotide polymorphism (SNP)” is a single base (nucleotide) polymorphism in a DNA sequence among individuals in a population. Typically in the literature, a single nucleotide polymorphism (SNP) may fall within coding sequences of genes, non-coding regions of genes, or in the intergenic regions between genes. SNPs within a coding sequence will not necessarily change the amino acid sequence of the protein that is produced, due to degeneracy of the genetic code. A SNP in which both forms lead to the same polypeptide sequence is termed “synonymous” (sometimes called a silent mutation)—if a different polypeptide sequence is produced they are “nonsynonymous”. A nonsynonymous change may either be missense or “nonsense”, where a missense change results in a different amino acid, while a nonsense change results in a premature stop codon.

Probes and primers: A probe comprises an isolated nucleic acid capable of hybridizing to a target nucleic acid. A detectable label or reporter molecule can be attached to a probe or primer. Typical labels include radioactive isotopes, enzyme substrates, co-factors, ligands, chemiluminescent or fluorescent agents, haptens, and enzymes. Methods for labeling and guidance in the choice of labels appropriate for various purposes are discussed, for example in Sambrook et al. (In Molecular Cloning: A Laboratory Manual, CSHL, New York, 1989) and Ausubel et al. (In Current Protocols in Molecular Biology, John Wiley & Sons, New York, 1998).

In a particular example, a probe includes at least one fluorophore, such as an acceptor fluorophore or donor fluorophore. For example, a fluorophore can be attached at the 5′- or 3′-end of the probe. In specific examples, the fluorophore is attached to the base at the 5′-end of the probe, the base at its 3′-end, the phosphate group at its 5′-end or a modified base, such as a T internal to the probe.

Probes are generally at least 15 nucleotides in length, such as at least 15, at least 16, at least 17, at least 18, at least 19, least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50 at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, or more contiguous nucleotides complementary to the target nucleic acid molecule, such as 20-70 nucleotides, 20-60 nucleotides, 20-50 nucleotides, 20-40 nucleotides, or 20-30 nucleotides.

Primers are short nucleic acid molecules, for instance DNA oligonucleotides are 10 nucleotides or more in length, which can be annealed to a complementary target nucleic acid molecule by nucleic acid hybridization to form a hybrid between the primer and the target nucleic acid strand. A primer can be extended along the target nucleic acid molecule by a polymerase enzyme. Therefore, primers can be used to amplify a target nucleic acid molecule.

The specificity of a primer increases with its length. Thus, for example, a primer that includes 30 consecutive nucleotides will anneal to a target sequence with a higher specificity than a corresponding primer of only 15 nucleotides. Thus, to obtain greater specificity, probes and primers can be selected that include at least 15, 20, 25, 30, 35, 40, 45, 50, 55, 60, 65, 70 or more consecutive nucleotides. In particular examples, a primer is at least 15 nucleotides in length, such as at least 15 contiguous nucleotides complementary to a target nucleic acid molecule. Particular lengths of primers that can be used to practice the methods of the present disclosure include primers having at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, at least 50, at least 55, at least 60, at least 65, at least 70, or more contiguous nucleotides complementary to the target nucleic acid molecule to be amplified, such as a primer of 15-70 nucleotides, 15-60 nucleotides, 15-50 nucleotides, or 15-30 nucleotides.

Primer pairs can be used for amplification of a nucleic acid sequence, for example, by PCR, real-time PCR, or other nucleic-acid amplification methods known in the art. An “upstream” or “forward” primer is a primer 5′ to a reference point on a nucleic acid sequence. A “downstream” or “reverse” primer is a primer 3′ to a reference point on a nucleic acid sequence. In general, at least one forward and one reverse primer are included in an amplification reaction.

Nucleic acid probes and primers can be readily prepared based on the nucleic acid molecules provided herein. It is also appropriate to generate probes and primers based on fragments or portions of these disclosed nucleic acid molecules, for instance regions that encompass the identified polymorphisms of interest. PCR primer pairs can be derived from a known sequence by using computer programs intended for that purpose such as Primer (Version 0.5, © 1991, Whitehead Institute for Biomedical Research, Cambridge, Mass.) or PRIMER EXPRESS® Software (Applied Biosystems, AB, Foster City, Calif.).

Sample: A sample, such as a biological sample, is a sample obtained from a subject. As used herein, biological samples include all clinical samples useful for establishing a skin profile for a subjects, including, but not limited to, cells, tissues, and bodily fluids (such as saliva); biopsied or surgically removed tissue, including tissues that are, for example, unfixed, frozen, fixed in formalin and/or embedded in paraffin; tears; skin scrapes; or surface washings. In a particular example, a sample includes cells collected by using a swab or by an oral rinse.

Subject: Living multi-cellular vertebrate organisms, a category that includes human and non-human mammals (such as laboratory or veterinary subjects).

### II. Skin Analysis Methods

Disclosed herein are skin analysis methods and assays which objectively identify biological strengths and weaknesses in a patient's genetic coding that affect the health and beauty of their skin. The disclosed methods and assays utilize a subject's unique genetic information to identify the genetic potential of the subject's skin in at least one area of skin health and thus reveal the subject's genetic predisposition to certain skin conditions and/or disorders. In each area of skin health, a score is generated (ranging from 0.00-1.00, with 1.00 being the optimal result) by analyzing a sample of the subject's DNA for genetic variations (or SNPs) that affect normal gene functioning. The number and location of these variations, determine the scores. The scores were determined by giving each SNP a numerical value based on such things as but not limited to population frequency and presence on one or two of each chromosomal pair affected as well as the number of SNP's affecting category. These scores were provided to provide the individual tested with a numerical score with which to compare this score. These scores and the detailed results present a personalized and highly preventive approach to skincare, giving a subject the opportunity for the earliest and best intervention possible before visible signs of skin deterioration or aging appear. In some examples, for calculating score a +1 may be given for every positive indicator on a chromosome at a particular location. Thus, if both chromosomal locations have the ideal allele a score of one may be given. If one chromosome has a negative coding and one does not, a score of 0.5 may be given and if both of the pair of chromosomes have the negative allele a score of 0.25 may be given.

In some examples, a personalized skin profile is generated by the following process:
1. Obtain genomic DNA sample, such as from from saliva, cheek swab, skin, or other tissue;
2. Isolate the genomic DNA from sample;
3. Verify the DNA quality and concentration;
4. Amplify the DNA;
5. Use the DNA as a template to detect/amplify the SNP regions for sequence determination;
6. Sequence the SNP regions to determine alleles/variants present;
7. Input the raw data into a processing database to verify sequences in human population;
8. Perform quality control check and input subject data into algorithms and group data analysis to determine scores by skin health category; and
9. Perform additional quality control check and generate final, user-friendly report for individual.
   It is contemplated that one or more of these steps may be repeated and/or omitted. It is also contemplated that the order of the steps may be altered.

In some examples, a DNA report is statistically tabulated and electronically created through a computer system. For example, this DNA report may include one or more of the following:

A list of the overall risk ratings of high, medium, or low under one or more categories of skin health and beauty, such as one, two, three, four, five or more, including up to 5 or greater than 5 categories of skin health and beauty;

A description of the biological effects and the visible signs of one or more categories of skin health and beauty, such as one, two, three, four, five or more, such as up to 5 or greater than 5 categories of skin health and beauty;

A page for each of the up to 5 or greater than 5 categories describing the category in greater detail and listing the marker-specific results for the individual is created. A rating system may include the following:
a) A value of 0.25 may be assigned to a result of homozygous for the non-ideal allele at the at risk SNP location (non-ideal function), which indicates high risk and is represented by a deficient rating;
b) A value of 0.50 may be assigned to a result of heterozygous for the non-ideal allele at the risk SNP location, which indicates medium risk and is represented by a sub-normal rating;
c) A value of 1.00 may be assigned to a result of homozygous for the ideal allele at the risk SNP location, which indicates low risk and is represented by a normal rating;
d) A computer program/algorithm calculates a numerical value for each category of skin health and beauty that may be based on the average of the numbers provided for each SNP contained in a skin health category and generates an overall numerical rating;
e) High risk may be considered an average numerical value of 0.25-0.5. Medium risk may be considered to be a numerical average of 0.5-0.75 and low risk may be considered to be a numerical value of 0.75-1.0; and
f) Marker specific results may be listed by SNP and may include:
   The affected gene or marker
   The chromosomal location
   The ideal genotype
   The actual genotype
   The rating.

In some examples, specific product recommendations are created according to a program based on each individual's report. Foundational products are recommended for everyone and these do not represent the unique product recommendations made for the individual based on their genetic results. These products are relevant; however, in that they set the stage for optimal results for personalized serums and supplements, based on each individual's unique DNA report. These foundational products may include:
a. An Antioxidant Cleanser
b. A Balancing Toner
c. An Antioxidant Moisturizer

In some examples, one or more pages describing the recommended personalized treatment serums and supplements based on the individuals high and medium risk category results are listed. For example, following recommendations for non-customized products, one or more custom-designed products may be recommended based on the DNA report. These recommendations may include a combination of treatment serums/nutritional products that address the individual strengths and weaknesses of the individuals skin as determined by their DNA report. These customized recommendations many include products such as:
a. Serums:
   1. Vitamin C Treatment Serum
   2. Hyaluronic Moisture Treatment Serum
   3. Wrinkle Treatment Serum
   4. Calming Treatment Serum
b. Supplements:
   1. Antioxidant Defense
   2. Glycation Defense
   3. Sun Defense
   4. Collagen Defense
   5. Inflammation Defense
      The determination as to which products will be custom recommended may be based on an algorithmic protocol that matches the ingredients in the products to the weakness that the person's DNA analysis reveals as identified in their medium or high risk categories. This may be programmed based on an algorithm into the computer system that generates the DNA report. High risk categories are an influencing category as recommendations to follow as they provide the individual the greatest opportunity to improve the health and appearance of their skin followed by the medium risk category recommendations. For instance, if a person is determined to be medium or high risk for collagen, products will be recommended that support healthy collagen production and maintenance in the skin. The ingredients in the products that support healthy collagen production and maintenance will be selected and put into the skin care products and nutritional supplements based on clinical research that supports the clinical effectiveness of the ingredients included in the product on the category to which it applies. Raw ingredient selection may be supported by clinical research regarding the effectiveness of the ingredient as identified in both peer-reviewed literature as well as clinical studies. These human clinical studies may be performed by a raw ingredient manufacturer to validate and support the effectiveness of the ingredient in well-controlled human trials. In some examples, product recommendations will be organized into high risk and medium risk categories on the report for ease of understanding and product selection by the individual. In some examples, if a product is already listed under a high-risk category, it will not be relisted under a medium risk category as it is already selected by the algorithm and recommended. In some examples, ingredients will be upgraded and changed on a regular basis based on the current science and literature as new ingredients with better product effectiveness become available in the raw ingredient marketplace.

In some examples, following the formulation process and algorithmic recommendations above, products may be clinically used and evaluated in a clinical practice to ensure that the effectiveness, aesthetic appeal and client satisfaction are of the highest standards possible.

It is contemplated that disclosed products may be distributed into the marketplace through various distribution models which may include direct to consumer, business to business, direct sales through a Multilevel marketing program, television/infomercial. In some examples, after reviewing the report, the report may be provided to the subject, either directly or indirectly (e.g., electronically or standard mail) and an optional customer service meeting/call is set up for those who desire to go over the results and product recommendations, and/or have questions regarding their report

In some embodiments, a method of characterizing a subject's skin is provided which includes generating a personalized skin profile. In some examples, generating a personalized skin profile includes determining a subject's genetic potential in at least one area of skin health or appearance by analyzing one or more skin health-associated single nucleotide polymorphisms (SNPs) or other genetic marker associated with the particular area of skin health or appearance being assessed in a biological sample obtained from the subject. In some examples, a subject's genetic potential is determined in one to five or more areas of skin health by analyzing one or more skin health-associated SNPs or other genetic markers associated with the one to five or more areas of skin health in a sample obtained from the subject. The one or more areas of skin health can include assessing the following or more factors: collagen formation, sun protection, antioxidant protection, glycation protection and inflammation control. The generated skin profile reveals the subject's genetic strengths, weaknesses and/or risks related to the one or more areas of skin health thereby allowing a personalized skincare and/or nutritional regimen to be developed and implemented. In some examples, a disclosed method of characterizing a subject's skin further includes identifying SNPs or other genetic markers associated with a particular area of skin health. For example, SNPs associated with a particular area of skin health can be identified by searching the publically available SNP on the Worldwide Web (see for example, domain name ncbi.nlm.nih.gov/snp; domain name ncbi.nlm.nih.gov/projects/SNP/; or domain name snp.cshl.org/) and determining SNPs associated with particular skin conditions. In some examples, a disclosed method of characterizing a subject's skin further includes providing the results of the characterization study to the subject. In some examples a disclosed method of characterizing a subject's skin further includes recommending and/or providing one or more skincare treatments to the subject based upon the skin profile generated by the characterization analysis. In some examples, the disclosed method of characterizing a subject's skin is performed at home. For example, a subject utilizes a kit designed to allow a subject to generate a skin profile by obtaining a DNA sample at home with the kit which includes an instruction booklet, a questionnaire, a DNA swab, a collection envelope with dessicant to place the specimen in after collection. This sample is then sent for analysis and evaluation and a report is generated for the individual.

In some examples, the kit includes a means for obtaining a biological sample, such a buccal swab and a collection vial which allows the sample to be stored during shipment to the analysis laboratory. The instructions for use can in any form, such as in a pamphlet or provided via electronic means, such as a website on the Worldwide Web.

In some examples, the disclosed method includes identifying one or more, such as one, two, three, four, five or more, categories of skin health and clinical studies to prove these categories have an impact on the skin (glycation causes aging, etc). For example, one or more SNPs is identified by identifying an SNP with an RS number that affects the enzyme or function in each category. Studies, such as clinical studies, are performed to identify the variations of the base at this location to validate that it is an SNP versus an infrequent variant and the clinical significance of this (it affects collagenase, etc.) Tests are then performed to identify which base patterns are protective versus risk promoting. The impact of the one or more SNPs identified on each skincare category is determined, such as by use of an algorithm. Additional studies are then performed to show that the disclosed treatments/protocols impact that area of skin health.

i. Obtaining a Biological Sample

Biological samples include all clinical samples useful for generating a skin profile for a subject, including, but not limited to, cells, tissues, and bodily fluids (such as saliva); biopsied or surgically removed tissue, including tissues that are, for example, unfixed, frozen, fixed in formalin and/or embedded in paraffin; tears; skin scrapes; or surface washings. In a particular example, a sample includes cells collected by using a buccal swab or by an oral rinse.

In some examples, a sample including nucleic acids is obtained from a subject who is suspected to have or develop a genetic predisposition to a skin condition, disorder or disease by a buccal swab. In some examples, the subject is displaying one or more signs or symptoms of skincare condition or disorder, such as skin redness, poor wound healing, accelerated aging, skin laxity and/or sagging, blemishes, excess pigmentation, freckles and/or brown spots, skin thinning, fine lines, rough skin surface texture, enlarged pores, broken capillaries, uneven skin tone, irregular pigmentation, acne, rosacea; excess skin dryness or oiliness, accelerated aging, thinning of skin, skin rashes, swelling and/or dermatitis (eczema), enhanced sensitivity to foreign substances like bacteria and chemicals, heavy wrinkles and/or skin folds, cracking skin, and/or uneven skin texture. In some examples, a sample is obtained from a subject that has family members who have or have had one or more skin disorders or diseases. In some examples, a sample is obtained from a subject that does not show any or minimal signs or symptoms of a skin care condition.

ii. Measuring Skin Health-Associated SNPs

The disclosed methods include measuring skin health-associated SNPs or other genetic markers in the biological sample obtained from the subject and comparing that to a control or reference value. In some examples, a subject's genetic potential is determined in five areas of skin health by analyzing one or more skin health-associated SNPs or other genetic markers associated with the five areas of skin health in a sample obtained from the subject. The one or more areas of skin health can include assessing the following factors: collagen formation, sun protection, antioxidant protection, glycation protection and inflammation control. Each of these factors is described in detail below. In some examples, a subject's genetic potential is determined in one or more areas of skin health by measuring one or more skin health-associated matrix metalloproteinase-1 (MMP1), glutathione peroxidase 1 (GPX1), glutathione peroxidase 2 (GPX2), leptin receptor (LEPR), agouti signaling protein (ASIP), ERCC2 (also known as XPD), human 8-oxoguanine DNA N-glycosylase (HOGG1), XRCC2, superoxide dismutase 1 (SOD1), superoxide dismutase 2 (SOD2), superoxide dismutase 3 (SOD3), epoxide hydrolase 1 (EPHX1), epoxide hydrolase 2 (EPX2), epoxide hydrolase 3 (EPX3), glutathione s-transferase pi (GSTP1), glutathione s-transferase pi pseudogene 1 (GSTP1P1), tumor necrosis factor alpha (TNFα), and/or nuclear factor erythroid 2-related 2 (NRF2L2) SNPs. In some examples, a subject's genetic potential is determined in one or more areas of skin health by measuring one or more skin health-associated SNPs with an RS number listed in Tables for 2 (each of these SNPs is incorporated herein by reference as available to the public such as on the Worldwide Web (see for example, domain name ncbi.nlm.nih.gov/snp; domain name ncbi.nlm.nih.gov/projects/SNP/; or domain name snp.cshl.org/) on Apr. 24, 2014), such as one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty three, thirty-four, thirty-five, thirty-six or more SNPS with an RS number listed in Tables 1 and/or Table 2.

Methods of isolating nucleic acid molecules from a biological sample are routine and known to those of ordinary skill in the art, for example using PCR to amplify the molecules from the sample, or by using a commercially available kit to isolate DNA. Nucleic acid molecules isolated from buccal swab samples or any other biological sample can be amplified using routine methods to form nucleic acid amplification products. Exemplary methods of isolating DNA and detecting SNPs associated with one or more skin conditions or disorders are described below in the Molecular Methods Section.

a. Collagen formation factor (ColFF)

Collagen is a principal structural protein of the skin and plays a role in skin firmness, fullness or plumpness and well as wrinkles. The speed of collagen synthesis and breakdown is influenced by a subject's genetic makeup. Genes known to be involved in slowing the breakdown and/or degradation of collagen fibers in skin can be collagen formation factors. A higher score in the disclosed assay of collagen formation factors indicates a more ideal genetic disposition for slowing the breakdown of collagen. A lower score indicates a greater likelihood of collagen breakdown, an MMP-1 and collagen imbalance, a decrease in tissue remodeling, ineffective wound healing and thus, the need for a skincare and/or nutritional treatment to prevent, inhibit or reduce one or more of these factors. A need for treating a collagen formation associated condition can also be identified by the presence of one or more of the following: prolonged skin redness; poor wound healing; accelerated aging; and/or skin laxity and/or sagging.

b. Sun Protection Factor (SunPF)

Ultraviolet (UV) exposure causes skin deterioration, premature skin aging, and a host of other profound changes to your skin. Exposure to UV light from the sun accounts for 90% of the symptoms of early skin aging. A subject's genetic makeup influences the effect that UV exposure on skin. A higher score with the present assay indicates a greater natural genetic protection. A lower score indicates likelihood of increased UV free radical damage, irregular cellular function, increased mitochondrial damage, DNA structural damage and ineffective melanogenesis and thus, the need for a skincare and/or nutritional treatment to prevent, inhibit or reduce one or more of these factors. A need for treating a sun protection associated condition can also be identified by the presence of one or more of the following: blemishes and redness; excess pigmentation, freckles and/or brown spots: skin thinning; fine lines; rough surface texture; enlarged pores; and/or redness/broken capillaries.

c. Antioxidant Protection Factor (AoxPF)

The oxidation phenomenon caused by free radicals is recognized as one of the leading causes of skin aging. A primary factor in determining damage from free radicals is controlled by genes known to be associated with antioxidant activities. A higher score indicates an increased genetic advantage for antioxidant protection. A lower score indicates likelihood of heightened free-radical cellular destruction, premature cell death; increased mitochondrial damage; decreased antioxidant functioning, decreased quinone detoxification and thus, the need for a skincare and/or nutritional treatment to prevent, inhibit or reduce one or more of these factors. A need for treating an antioxidant protection associated condition can also be identified by the presence of one or more of the following: uneven skin tone, irregular pigmentation; rough skin texture; acne and rosacea; excess skin dryness or oiliness; and/or accelerated aging and/or thinning of skin.

d. Glycation Protection Factor (GlyPF)

Advanced Glycation End Products (AGEs) are the end result of a glucose-driven process known as glycation. Glycation is implicated in accelerated skin aging, leading to wrinkling, dryness, sagging, and laxity in your skin. A subject's score indicates the subject's genetic protection against glycation: a higher score indicates a more optimal predisposition. A lower score indicates likelihood of glucose/collagen cross-linking: decreased skin elasticity; stiffened collagen fibers, weak dermal epidermal junctions; increased production of free radicals, and thus, the need for a skincare and/or nutritional treatment to prevent, inhibit or reduce one or more of these factors. A need for treating a glycation protection associated condition can also be identified by the presence of one or more of the following: heavy wrinkles and/or skin folds; accelerated aging; sagging skin; cracking and thinning skin; and/or uneven skin texture.

e. Inflammation Control Factor (InfCF)

Inflammation is skin's first line of defense against foreign substances like bacteria and chemicals. However, excessive inflammation is one of the most common causes of early onset skin deterioration and aging. A subject's genetic makeup play a role in the regulation of inflammation: a higher score indicates a greater capacity to reduce inflammation.

A lower score indicates possible irregular tissue healing, decreased cellular defense, overactive inflammatory signaling, enhanced sensitivity, decreased efficacy of the detoxification process, increased production of free radicals and thus, the need for a skincare and/or nutritional treatment to prevent, inhibit or reduce one or more of these factors. A need for treating an inflammation control factor associated condition can also be identified by the presence of one or more of the following: skin redness; acne rosacea; rashes, swelling and/or dermatitis (eczema); accelerated aging; and/or enhanced sensitivity to foreign substances like bacteria and chemicals.

iii. Providing a Skin Profile to a Subject

Following the measurement of one or more SNPs associated with skin health, the results, findings, diagnoses, predictions and/or treatment recommendations can be provided to the subject. For example, the results, findings, diagnoses, predictions and/or treatment recommendations can be recorded and communicated to technicians, physicians and/or patients or clients. In certain embodiments, computers can be used to communicate such information to interested parties, such as, clients, patients and/or the attending physicians. Based on the measurement, the therapy or protocol administered to a subject can be started, modified not started or re-started (in the case of monitoring for a reoccurrence of a particular skin condition/disorder).

In some examples, the output can provide a recommended therapeutic regimen or skin care protocol. In some examples, the test may include determination of other clinical information.

In some embodiments, the disclosed methods include one or more of the following depending on the subject's skin profile: a) prescribing or recommending a protocol or treatment regimen for the subject if the subject's determined profile is considered to be high or medium risk, sub-optimal or deficient in one or more areas of skin health; b) not prescribing or recommending a protocol or treatment regimen for the subject if the subject's determined skin profile is considered to be optimal in the evaluated skin areas; c) administering a protocol or treatment to the subject if the subject's determined diagnosis or profile is considered to be high or medium risk or sub-optimal or deficient in one or more areas of skin health or appearance; d) not administering a protocol or treatment regimen to the subject if the subject's determined skin profile is considered to be optimal in the evaluated skin areas. In an alternative embodiment, the method can include recommending one or more of a)-d).

In one embodiment, a diagnosis, prediction and/or treatment recommendation or protocol based on the skin profile disclosed herein is communicated to the subject as soon as possible after the assay is completed and the diagnosis and/or prediction is generated. The results and/or related information may be communicated to the subject by the subject's treating physician. Alternatively, the results may be communicated directly to a test subject by any means of communication, including writing, such as by providing a written report, electronic forms of communication, such as email, or telephone. Communication may be facilitated by use of a computer, such as in case of email communications. In certain embodiments, the communication containing results of a skin profile analysis and/or conclusions drawn from and/or treatment recommendations or protocols based on the test, may be generated and delivered automatically to the subject using a combination of computer hardware and software which will be familiar to artisans skilled in telecommunications. One example of a healthcare-oriented communications system is described in U.S. Pat. No. 6,283,761; however, the present disclosure is not limited to methods which utilize this particular communications system. In certain embodiments of the methods of the disclosure, all or some of the method steps, including the assaying of samples, performing the comparisons, and/or communicating of assay results, diagnoses or recommendations, may be carried out in diverse (e.g., foreign) jurisdictions.

In several embodiments, identification of a subject as having or at risk of developing a skincare condition or disorder results in the physician treating the subject, such as prescribing one or more therapeutic agents for inhibiting or delaying one or more signs and symptoms associated with the disorder/condition. In additional embodiments, the treatment, dose or dosing regimen is modified based on the information obtained using the methods disclosed herein.

The subject can be monitored while undergoing treatment using the methods described herein in order to assess the efficacy of the treatment protocol. In this manner, the length of time or the amount given to the subject can be modified based on the results obtained using the methods disclosed herein. The subject can also be monitored after the treatment using the methods described herein to monitor for relapse and thus, the effectiveness of the given treatment. In this manner, whether to resume treatment can be decided based on the results obtained using the methods disclosed herein. In some examples, this monitoring is performed by a clinical healthcare provider.

In some embodiments, once a subject's skin profile is determined, an indication of that profile can be displayed and/or conveyed to a clinician or other caregiver. For example, the results of the test are provided to a user (such as a clinician or other health care worker, laboratory personnel, or patient) in a perceivable output that provides information about the results of the test. In some examples, the output is a paper output (for example, a written or printed output), a display on a screen, a graphical output (for example, a graph, chart, or other diagram), or an audible output.

In other examples, the output is a numerical value, such as an amount of a particular set of SNPs or other genetic markers in the sample as compared to a control. In additional examples, the output is a graphical representation, for example, a graph that indicates the value (such as amount or relative amount) of the set of SNPs in the sample from the subject on a standard curve. In a particular example, the output (such as a graphical output) shows or provides a cut-off value or level that indicates the presence of optimal, sub-optimal or deficient skin factor level. In some examples, the output is communicated to the user, for example by providing an output via physical, audible, or electronic means (for example by mail, telephone, facsimile transmission, email, or communication to an electronic medical record).

The output can provide quantitative information (for example, an amount of an molecule in a test sample compared to a control sample or value) or can provide qualitative information (for example, a diagnosis of a deficiency in collagen formation factors, sun protection factors, antioxidant protection factors, glycation protection factors and/or inflammation control factors). In additional examples, the output can provide qualitative information regarding the relative amount of a particular SNP in the sample, such as identifying presence of an increase relative to a control, a decrease relative to a control, or no change relative to a control.

In some examples, the output is accompanied by guidelines for interpreting the data, for example, numerical or other limits that indicate the presence or absence of a skincare disorder/condition. The indicia in the output can, for example, include normal or abnormal ranges or a cutoff, which the recipient of the output may then use to interpret the results, for example, to arrive at a diagnosis, prognosis, susceptibility towards or treatment plan.

iv. Providing Skincare Treatment to a Subject

In some embodiments, the method further includes providing an appropriate therapy or protocol for the subject diagnosed with a skincare disorder/condition. In some examples, the therapy includes administering an agent to alter one or more signs or symptoms associated with the identified skincare disorder/condition. In some examples, one or more of the agents described below are administered to a subject to treat or support one or more sub-normal or deficient skin factors or medium or high risk areas identified by the disclosed skin profile. It is contemplated that the desired treatments or protocols may be administered via any means known to one of skill in the art, including oral and/or topical administration. In some examples, a composition is administered to the subject orally, such as in a capsule or tablet. In some examples, a composition is administered topically. It is contemplated that one or more compositions can be administered via multiple routes as the same or different time period depending upon the disorders/conditions being treated. In some examples, the composition is one or more of the compositions disclosed below. It is contemplated that compositions with similar properties could be administered as well.

a. Types of Skin Surface to Treat

Any skin surface can be treated using the methods provided herein. By “skin surface” is intended the stratum corneum, epidermis, dermis or any other layer of the skin thereof. Skin surfaces that can be treated include, but are not limited to face, scalp, neck, chest, back, torso, arms, legs, hands or feet including periorbits, lips, cheeks, nasolabial folds, forehead, chin, neck, upper lip rhytides, or any combination thereof. The skin of any facial surface can be treated using the methods provided herein. The method can be applied to any facial or scalp area and/or to any body surface area, with other immediate areas of application being the chest, neck and body. More than one skin surface can be treated during the same treatment period.

Improving skin quality includes reversing, slowing the progression of, supporting the healthy function of or preventing skin changes associated with natural or innate aging or other biological or disease effects. As used herein, “prevent” and variations thereof refer to any degree of delaying the onset of skin changes. For example, improving skin quality includes the reversal, slowing the progression of, or prevention of skin changes associated with sun damage or photo aging, skin changes associated with exposure to sunlight or other forms of actinic radiation (for example, UV radiation and tanning booths). As another example, improving skin quality also can include reversing, slowing the progression of, or preventing skin changes resulting from extrinsic factors, including, but not limited to, radiation, air pollution, sun, UV rays, wind, cold, dampness, heat, chemicals, smoke, cigarette smoking, and combinations thereof. Improving skin quality also can include reversing, preventing or reducing scarring the can result, for example, from certain skin conditions (for example, acne), infections (for example, leishmaniasis), or injury (for example, abrasions, punctures, lacerations, or surgical wounds). Improvements to the skin can also include at least one of the following: reducing red, brown or any other abnormal pigment making facial lines appear less noticeable, making facial lines and/or wrinkles feel plumped, improving the appearance of suborbital lines and/or periorbital lines, improving the appearance of crow's feet, reducing and/or diminishing the appearance of wrinkles, particularly facial wrinkles on the cheeks, forehead (for example, perpendicular wrinkles between eyes, horizontal wrinkles above the eyes), and/or around the mouth, and particularly deep wrinkles, folds, or creases, improving skin suppleness smoothness texture and tone, reducing and/or eliminating fine and/or deep lines, folds and creases, and smoothing skin. Methods for measuring improved skin quality are known in the art. See, for example, U.S. Pat. Nos. 6,866,856 and 6,682,763.

Skin changes treatable by practicing the methods and using the assays disclosed herein include, for example, wrinkles (including, but not limited to, human facial wrinkles), creases, furrows, folds and fine lines, deepening of skin lines, thinning of skin, reduced scarring, yellowing, browning or reddening of the skin, mottling, hyperpigmentation, appearance of pigmented and/or non-pigmented age spots, leatheriness, loss of elasticity, loss of recoilability, loss of collagen fibers, abnormal changes in the elastic fibers, deterioration of small blood vessels of the dermis, formation of solar increased visible vasculature on the skin surface, inflammation including redness, dryness or irritation of the skin or any other skin abnormality or combinations thereof.

Improving skin quality includes decreasing, reducing, and/or minimizing one or more of the skin changes discussed above. Improving skin quality can result in the skin having a more youthful and healthy appearance. Improving skin quality can result in the skin having a smoother, hydrated (less dry), or less scaly appearance. For example, in certain embodiments, improving skin quality can include a reduction in roughness, dryness, irritation or scaliness. Improving skin quality includes the effacement and improvement of lines and wrinkles, improvement in turgor, and tonicity, with the observed desired effects of lifting and tightening.

The textural qualities of the skin can be improved, including softness, suppleness, and smoothness, leading to enhancement of luster, clarity and brightness. Additional and important qualities of the skin that can be subjectively and objectively measured include, but are not limited to skin laxity, or conversely skin tightness, and the presence and degree of textural fine lines and coarser lines within the skin.

These are the same qualities by which the external aspects of appearance (for example, aging of skin) are judged. Improvement in these qualities by the method of treatment and kits disclosed herein result in a benefit based on visual judgment of appearance. Changing a quality of the skin by the methods disclosed herein lessens the appearance of aging of the skin.

Desired benefits may include not only physiologic benefit to the skin, but therapeutic and pharmacologic benefits, such as possible malignancy prevention and treatment. Benefits may also include acne treatment and suppression, by including compositions which suppress sebaceous glandular activity, enhance bacterial suppression, or enhance retinoid delivery into the skin.

The percentage of improvement can be, for example, at least about 20%, at least about 30%, at least about 40%, at least about 50%, at least about 60%, at least about 70%, at least about 80%, at least about 90% or at least about 100% compared to the baseline score prior to treatment with one or more skin agents including those described herein. The improvement can be measured by both subjective and objective methods, and can be quantified using a subjective scoring or a panel scoring, amongst other methods. By “baseline” is intended the score prior to treatment or the score on an untreated area of skin.

The treatment can be performed multiple times for optimal results. In one embodiment, the treatment is performed twice a day. In another embodiment, the treatment is performed daily. In other embodiments, the treatment is performed weekly. In another embodiment, the treatment is performed monthly. In another embodiment, the treatment is performed at least once every one to two days. In another embodiment, the treatment is performed at least once every one to two weeks. In other embodiments, the treatment is performed as described below with one or more of the disclosed compositions.

b. Exemplary Compositions

i. Antioxidant Cleanser

In some examples, an antioxidant cleanser is applied topically to a skin surface as needed, such as to prevent, reduce, or inhibit one or more signs or symptoms associated with high or medium risk or sub-normal or deficient skin health factors. In one example, the antioxidant cleanser comprises: sodium lauryl glucose carboxylate; lauryl glucoside; coco-glucoside; cocamidopropyl betaine; glyceryl oleate; glycerin; caprylyl glycol; citrus grandis (grapefruit) peel oil; panthenol; solanum lycopersicum (tomato) extract; citrus aurantium bergamia (bergamot) fruit oil; thymus vulgaris (thyme) extract; algae extract; aloe barbadensis leaf; and citrus medica limonum (lemon) peel oil. In some examples, the antioxidant cleanser is commercially available SKINSHIFT® Antioxidant Cleanser Formula 112.

ii. Balancing Toner

In some examples, a balancing toner is applied topically to a skin surface as needed, such as to prevent, reduce, or inhibit one or more signs or symptoms associated with high or medium risk or sub-normal or deficient skin health factors. In one example, the balancing toner comprises: methyl gluceth-20; glycerin; caprylyl glycol; sodium PGA; panthenol; citrus grandis (grapefruit) peel oil; thymus vulgaris (thyme extract); citrus aurantium amara (bitter orange) extract; hamamelis virginiana (witch hazel) bark/leaf/twig extract; glycine soja (soybean) seed extract; solanum lycopersicum (tomato) extract; citrus aurantium bergamia (bergamot) fruit oil; aloe barbadensis leaf; ascorbic acid; tocopheryl acetate; retinyl palmitate; and bioflavonoids. In some examples, the antioxidant cleanser is commercially available SKINSHIFT® Balancing Toner Formula 121.

iii. Wrinkle Treatment Serum

In some examples, a wrinkle treatment serum is applied topically to a skin surface as needed, such as to prevent, reduce, or inhibit one or more signs or symptoms associated with high or medium risk or sub-normal or deficient skin health factors. In one example, the wrinkle treatment serum comprises: acetyl octapeptide-3 (SNAP-8); capryly glycol; glycerin; palmitoyl oligopeptide, palmitoyl tetrapeptide-7 (also known as Matrixyl-300); hyaluronic acid; solanum lycopersicum (tomato) extract; thymus vulgaris (thyme) extract; aloe barbadensis leaf; leontopodium alpinum (edelweiss) flower/leaf extract; glycine soja (soybean) seed extract; citrus grandis (grapefruit) peel oil; and allantoin. In some examples, the antioxidant cleanser is commercially available SKINSHIFT® Wrinkle Treatment Serum Formula 132.

iv. Vitamin C Treatment Serum

In some examples, a vitamin C treatment serum is applied topically to a skin surface as needed, such as to prevent, reduce, or inhibit one or more signs or symptoms associated with high or medium risk or sub-normal or deficient skin health factors. In one example, the vitamin C treatment serum comprises: cyclopentasiloxane, dimethicone crosspolymer, cyclomethicone; sodium ascorbyl phosphate; idebenone; caprylyl glycol; citrus grandis (grapefruit) peel oil; magnesium ascorbyl phosphate; citrus aurantium dulcis (orange) peel oil; aloe barbadensis leaf; retinyl palmitate; and tocopherol. In some examples, the antioxidant cleanser is commercially available SKINSHIFT® Vitamin C Treatment Serum Formula 137.

v. Calming Treatment Serum

In some examples, calming treatment serum is applied topically to a skin surface as needed, such as to prevent, reduce, or inhibit one or more signs or symptoms associated with high or medium risk or sub-normal or deficient skin health factors. In one example, the calming treatment serum comprises: glycerin; sodium cocoyl amino acids, sarcosine, potassium aspartate, magnesium aspartate, caprylyl glycol; hyaluronic acid; caprylic/capric triglyceride; citrus grandis (grapefruit) peel oil; glycine soja (soybean) seed extract; thymus vulgaris (thyme) extract; arnica montana flower extract; solanum lycopersicum (tomato) extract; leontopodium alpinum (edelweiss) flower/leaf extract; ale barbadensis leaf; and epilobium angustifolium (canadian willow) extract. In some examples, the antioxidant cleanser is commercially available SKINSHIFT® Calming Treatment Serum Formula 131.

vi. Hyaluronic Moisture Treatment Serum

In some examples, hyaluronic moisture treatment serum is applied topically to a skin surface as needed, such as to prevent, reduce, or inhibit one or more signs or symptoms associated with high or medium risk or sub-normal or deficient skin health factors. In one example, the hyaluronic moisture treatment serum comprises: hyaluronic acid; sodium PGA; caprylyl glycol; glycerin; panthenol; avena sative (oat) kernel extract; thymus vulgaris (thyme) extract; saccharomyces/silicon ferment, saccharomyces/copper ferment, saccharomyces/iron ferment, saccharomyces/zinc ferment; aloe barbadensis leaf; leontopodium alpinum flower/leaf extract; and allantoin. In some examples, the antioxidant cleanser is commercially available SKINSHIFT® Hyaluronic Moisture Treatment Serum Formula 139.

vii. Antioxidant Moisturizer

In some examples, an antioxidant moisture is applied topically to a skin surface as needed, such as to prevent, reduce, or inhibit one or more signs or symptoms associated with high or medium risk or sub-normal or deficient skin health factors. In one example, the antioxidant moisture comprises: polyacrylamide; glycerin; sorbitol, caprylic/capric triglyceride, squalene, cyclomethicone, caprylyl glycol, hyaluronic acid, glycoproteins, citrus grandis, thymus vulgaris (thyme) extract, thioctic acid, solanum lycopersicum (Tomato) extract, camellia oleifera (green tea) leaf extract, oryza sativa (rice) bran oil, tocopheryl acetate, aloe barbadensis leaf, ubiquinone, and glycine soja (soybean) seed extract. In some examples, the antioxidant moisturizer is commercially available SKINSHIFT® Antioxidant Moisturizer Formula 142.

viii. Collagen Defense Composition

In some examples, a subject determined to have sub-normal or deficient levels of collagen protection factors, is administered a collagen defense composition to reduce, inhibit, and/or prevent one or more signs associated with high or medium risk or sub-normal or deficient levels of collagen protection factors. In some examples, the collagen defense composition is in tablet/capsule form and is administered to the subject twice daily and includes the following: Choline (as Choline-Stabilized Orthosilicic Acid), and Silicon (as Choline-Stabilized Orthosilicic Acid). In some examples, the collagen defense composition is commercially available SKINSHIFT® Collagen Defense.

ix. Sun Defense Composition

In some examples, a subject determined to have sub-normal or deficient levels of sun protection factors, is administered a sun defense composition to reduce, inhibit, and/or prevent one or more signs associated with high or medium risk or sub-normal or deficient levels of sun protection factors. In some examples, a sun defense compositions includes trans-resveratrol and quercetin. In some examples, the sun defense composition is in tablet/capsule form and is administered to the subject twice daily and includes the following: Trans-Resveratrol (Polygonum Cuspidatum), Quercetin Dihydrate, Lecithin, Microcrystalline Cellulose, Dicalcum Phosphate, Silicon Dioxide, Vegetable Stearate. In some examples, the sun defense composition is commercially available SKINSHIFT® Sun Defense.

x. Antioxidant Defense Composition

In some examples, a subject determined to have sub-normal or deficient levels of antioxidant protection factors, is administered an antioxidant defense composition to reduce, inhibit, and/or prevent one or more signs associated with high or medium risk or sub-normal or deficient levels of antioxidant protection factors. In some examples, the antioxidant defense composition is in tablet/capsule form and is administered to the subject three times daily and includes the following: Vitamin A (as Natural Mixed Carotenoids Complex), Alpha Carotene 2.5, Beta Carotene, Acerola (Malpighia Glabra), High Gamma Mixed Tocopherols, Grape Seed Extract (Wis Vinifera), Curcumin C3 Complex® (Curcumin, Bisdemethoxy Curcumin, Demethoxy Curcumin), Garlic (Allium Sativum), Tocotrienols (from Annatto Bean), Ginkgo Biloba, Quercetin, Rutin, Clove (Syzygium Aromaticum), Allspice (Pimenta Dioca), Sweet Basil (Ocimum Basilicum), Sage (Salvia Officinalis), Rosemary (Rosemarinus Officinalus), Polygonum Cuspidatum (50% Trans-Resveratrol), Lutein (Tagetes Erecta L) (Marigold Lutein Esters), Lycopene, Microcrystalline Cellulose, Silicon Dioxide, Stearates (Vegetable Source). In some examples, the antioxidant defense composition is commercially available SKINSHIFT® Antioxidant Defense.

xi. Glycation Defense Composition

In some examples, a subject determined to have sub-normal or deficient levels of glycation protection factors, is administered a glycation defense composition to reduce, inhibit, and/or prevent one or more signs associated with high or medium risk or sub-normal or deficient levels of glycation protection factors. Excess sugar in the body is a primary cause of premature skin aging because of its role in a process called glycation. Glycation occurs when blood sugar binds to collagen and elastin fibers, essentially “carmelizing” or hardening skin. Glycated skin results in skin laxity, cracking, thinning, redness and inability to self-repair. In some examples, a glycation defense composition is administered to the subject wherein the composition comprises herbs, polyphenols and antioxidants to sugar levels and protect against glycation. In some examples, the glycation defense composition is in tablet/capsule form and is administered to the subject twice daily and includes Salacia (Salacia Oblonga), Fennugreek (Trigonella Foenum-Graecum), American Ginseng (Panax Quinquefolius), Gymnema (Gymnema Sylvestre), Banaba (Langerstroemia Spp.), Kudzu (Pueraraia lobata), Cinnamon (Cinnamomum Spp.), Microcrystalline Cellulose, and Vegetable Stearate. In some examples, the glycation defense composition is commercially available SKINSHIFT® Glycation Defense.

xii. Inflammation Defense Composition

In some examples, a subject determined to have high or medium risk or sub-normal or deficient levels of inflammation protection factors, is administered an inflammation defense composition to reduce, inhibit, and/or prevent one or more signs associated with skin inflammation such as skin sensitivity, redness, irritation, acne, rosacea and eczema. In some examples, an inflammation defense composition comprises n*zimes® Proprietary Blend (Protease 6.0, Protease 4.5, Trypsin 1:150), Serrazimes®, Chymotrypsin, Turmeric (Curcuma Longa), Boswellia (Boswellia Serrata), Ginger (Zingiber Offinale), Ouercetin, Rutin, Rosemary Extract (Rosemarinus Officinalis), Microcrystalline Cellulose, Silicon Dioxide, Vegetable Stearate. This inflammation defense composition is administered in tablet/capsule form, twice daily. In some examples, the inflammation defense composition is commercially available SKINSHIFT® Inflammation Defense.

### Molecular Methods

Generally, the methods disclosed herein involve an assessment of nucleic acid sequence. Molecular techniques of use in all of these methods are disclosed below and in the Example Section.

Preparation of Nucleic Acids for Analysis:

Nucleic acid molecules can be prepared for analysis using any technique known to those skilled in the art. Generally, such techniques result in the production of a nucleic acid molecule sufficiently pure to determine the presence or absence of one or more variations at one or more locations in the nucleic acid molecule. Such techniques are described for example, in Sambrook, et al., Molecular Cloning: A Laboratory Manual (Cold Spring Harbor Laboratory, New York) (1989), and Ausubel, et al., Current Protocols in Molecular Biology (John Wiley and Sons, New York) (1997), incorporated herein by reference.

When the nucleic acid of interest is present in a cell, it can be necessary to first prepare an extract of the cell and then perform further steps, such as differential precipitation, column chromatography, extraction with organic solvents and the like, in order to obtain a sufficiently pure preparation of nucleic acid. Extracts can be prepared using standard techniques in the art, for example, by chemical or mechanical lysis of the cell. Extracts then can be further treated, for example, by filtration and/or centrifugation and/or with chaotropic salts such as guanidinium isothiocyanate or urea or with organic solvents such as phenol and/or HCCl₃ to denature any contaminating and potentially interfering proteins. When chaotropic salts are used, it can be desirable to remove the salts from the nucleic acid-containing sample. This can be accomplished using standard techniques in the art such as precipitation, filtration, size exclusion chromatography and the like.

In some instances, messenger RNA can be extracted from cells. Techniques and material for this purpose are known to those skilled in the art and can involve the use of oligo dT attached to a solid support such as a bead or plastic surface. In some embodiments, the mRNA can be reversed transcribed into cDNA using, for example, a reverse transcriptase enzyme. Suitable enzymes are commercially available from, for example, Invitrogen, Carlsbad Calif. Optionally, cDNA prepared from mRNA can also be amplified.

Amplification of Nucleic Acid Molecules:

Optionally, the nucleic acid samples obtained from the subject are amplified prior to detection. Target nucleic acids are amplified to obtain amplification products, including sequences from a haplotype block including a tag SNP, can be amplified from the sample prior to detection. Typically, DNA sequences are amplified, although in some instances RNA sequences can be amplified or converted into cDNA, such as by using RT PCR.

Any nucleic acid amplification method can be used. An example of in vitro amplification is the polymerase chain reaction (PCR), in which a biological sample obtained from a subject is contacted with a pair of oligonucleotide primers, under conditions that allow for hybridization of the primers to a nucleic acid molecule in the sample. The primers are extended under suitable conditions, dissociated from the template, and then re-annealed, extended, and dissociated to amplify the number of copies of the nucleic acid molecule. Other examples of in vitro amplification techniques include quantitative real-time PCR, strand displacement amplification (see U.S. Pat. No. 5,744,311); transcription-free isothermal amplification (see U.S. Pat. No. 6,033,881); repair chain reaction amplification (see PCT Publication NO. WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134).

In specific examples, the target sequences to be amplified from the subject include a nucleotide sequence of interest including the SNP. In an embodiment, a single SNP with exceptionally high predictive value is amplified, or a nucleic acid encoding the SNP is amplified.

A pair of primers can be utilized in the amplification reaction. One or both of the primers can be labeled, for example with a detectable radiolabel, fluorophore, or biotin molecule. The pair of primers includes an upstream primer (which binds 5′ to the downstream primer) and a downstream primer (which binds 3′ to the upstream primer). The pair of primers used in the amplification reactions are selective primers which permit amplification of a size related marker locus. Numerous primers can be designed by those of skill in the art simply by determining the sequence of the desired target region, for example, using well known computer assisted algorithms that select primers within desired parameters suitable for annealing and amplification.

If desired, an additional pair of primers can be included in the amplification reaction as an internal control. For example, these primers can be used to amplify a “housekeeping” nucleic acid molecule, and serve to provide confirmation of appropriate amplification. In another example, a target nucleic acid molecule including primer hybridization sites can be constructed and included in the amplification reactor. One of skill in the art will readily be able to identify primer pairs to serve as internal control primers.

Primer Design Strategy:

Increased use of polymerase chain reaction (PCR) methods has stimulated the development of many programs to aid in the design or selection of oligonucleotides used as primers for PCR. Four examples of such programs that are freely available via the Internet are: PRIMER™ by Mark Daly and Steve Lincoln of the Whitehead Institute (UNIX, VMS, DOS, and Macintosh), Oligonucleotide Selection Program by Phil Green and LaDeana Hiller of Washington University in St. Louis (UNIX, VMS, DOS, and Macintosh), PGEN™ by Yoshi (DOS only), and Amplify by Bill Engels of the University of Wisconsin (Macintosh only). Generally these programs help in the design of PCR primers by searching for bits of known repeated-sequence elements and then optimizing the Tₘ by analyzing the length and GC content of a putative primer. Commercial software is also available and primer selection procedures are rapidly being included in most general sequence analysis packages.

Designing oligonucleotides for use as either sequencing or PCR primers to detect requires selection of an appropriate sequence that specifically recognizes the target, and then testing the sequence to eliminate the possibility that the oligonucleotide will have a stable secondary structure. Inverted repeats in the sequence can be identified using a repeat-identification or RNA-folding programs. If a possible stem structure is observed, the sequence of the primer can be shifted a few nucleotides in either direction to minimize the predicted secondary structure. When the amplified sequence is intended for subsequence cloning, the sequence of the oligonucleotide can also be compared with the sequences of both strands of the appropriate vector and insert DNA. A sequencing primer only has a single match to the target DNA. It is also advisable to exclude primers that have only a single mismatch with an undesired target DNA sequence. For PCR primers used to amplify genomic DNA, the primer sequence can be compared to the sequences in the GENBANK™ database to determine if any significant matches occur. If the oligonucleotide sequence is present in any known DNA sequence or, more importantly, in any known repetitive elements, the primer sequence should be changed.

Detection of Alleles:

The nucleic acids obtained from the sample can be genotyped to identify the particular allele present for a marker locus. A sample of sufficient quantity to permit direct detection of marker alleles from the sample can be obtained from the subject. Alternatively, a smaller sample is obtained from the subject and the nucleic acids are amplified prior to detection. Any target nucleic acid that is informative for a chromosome haplotype can be detected. Any method of detecting a nucleic acid molecule can be used, such as hybridization and/or sequencing assays.

Hybridization is the binding of complementary strands of DNA, DNA/RNA, or RNA. Hybridization can occur when primers or probes bind to target sequences such as target sequences within genomic DNA. Probes and primers that are useful generally include nucleic acid sequences that hybridize (for example under high stringency conditions) with a nucleic acid sequence including the SNP of interest, but do not hybridize to a reference allele, or that hybridize to the reference allele, but do not hybridize to the SNP. Physical methods of detecting hybridization or binding of complementary strands of nucleic acid molecules, include but are not limited to, such methods as DNase I or chemical footprinting, gel shift and affinity cleavage assays, Southern and Northern blotting, dot blotting and light absorption detection procedures. The binding between a nucleic acid primer or probe and its target nucleic acid is frequently characterized by the temperature (Tₘ) at which 50% of the nucleic acid probe is melted from its target. A higher (Tₘ) means a stronger or more stable complex relative to a complex with a lower (Tm).

Generally, complementary nucleic acids form a stable duplex or triplex when the strands bind, (hybridize), to each other by forming Watson-Crick, Hoogsteen or reverse Hoogsteen base pairs. Stable binding occurs when an oligonucleotide molecule remains detectably bound to a target nucleic acid sequence under the required conditions.

Complementarity is the degree to which bases in one nucleic acid strand base pair with the bases in a second nucleic acid strand. Complementarity is conveniently described by percentage, that is, the proportion of nucleotides that form base pairs between two strands or within a specific region or domain of two strands. For example, if 10 nucleotides of a 15-nucleotide oligonucleotide form base pairs with a targeted region of a DNA molecule, that oligonucleotide is said to have 66.67% complementarity to the region of DNA targeted.

In the present disclosure, “sufficient complementarity” means that a sufficient number of base pairs exist between an oligonucleotide molecule and a target nucleic acid sequence (such as an SNP associated with a particular skin factor) to achieve detectable and specific binding. When expressed or measured by percentage of base pairs formed, the percentage complementarity that fulfills this goal can range from as little as about 50% complementarity to full (100%) complementary. In general, sufficient complementarity is at least about 50%, for example at least about 75% complementarity, at least about 90% complementarity, at least about 95% complementarity, at least about 98% complementarity, or even at least about 100% complementarity. The qualitative and quantitative considerations involved in establishing binding conditions that allow one skilled in the art to design appropriate oligonucleotides for use under the desired conditions is provided by Beltz et al. Methods Enzymol 100:266-285, 1983, and by Sambrook et al. (ed.), Molecular Cloning: A Laboratory Manual, 2nd ed., vol. 1-3, Cold Spring Harbor Laboratory Press, Cold Spring Harbor, N. Y., 1989.

Hybridization conditions resulting in particular degrees of stringency will vary depending upon the nature of the hybridization method and the composition and length of the hybridizing nucleic acid sequences. Generally, the temperature of hybridization and the ionic strength (such as the Na+ concentration) of the hybridization buffer will determine the stringency of hybridization. Calculations regarding hybridization conditions for attaining particular degrees of stringency are discussed in Sambrook et al., (1989) Molecular Cloning: a laboratory manual, second edition, Cold Spring Harbor Laboratory, Plainview, N.Y. (chapters 9 and 11). The following is an exemplary set of hybridization conditions and is not limiting:

Very High Stringency (Detects Sequences that Share at Least 90% Complementarity)

Hybridization: 5×SSC at 65° C. for 16 hours

Wash twice: 2×SSC at room temperature (RT) for 15 minutes each

Wash twice: 0.5×SSC at 65° C. for 20 minutes each

High Stringency (Detects Sequences that Share at Least 80% Complementarity)

Hybridization: 5×-6×SSC at 65° C.-70° C. for 16-20 hours

Wash twice: 2×SSC at RT for 5-20 minutes each

Wash twice: 1×SSC at 55° C.-70° C. for 30 minutes each

Low Stringency (Detects Sequences that Share at Least 50% Complementarity)

Hybridization: 6×SSC at RT to 55° C. for 16-20 hours

Wash at least twice: 2×-3×SSC at RT to 55° C. for 20-30 minutes each.

Methods for labeling nucleic acid molecules so they can be detected are well known. Examples of such labels include non-radiolabels and radiolabels. Non-radiolabels include, but are not limited to an enzyme, chemiluminescent compound, fluorescent compound (such as FITC, Cy3, and Cy5), metal complex, hapten, enzyme, colorimetric agent, a dye, or combinations thereof. Radiolabels include, but are not limited to, ¹²⁵I, ³²P and ³⁵S. For example, radioactive and fluorescent labeling methods, as well as other methods known in the art, are suitable for use with the present disclosure. In one example, primers used to amplify the subject's nucleic acids are labeled (such as with biotin, a radiolabel, or a fluorophore). In another example, amplified target nucleic acid samples are end-labeled to form labeled amplified material. For example, amplified nucleic acid molecules can be labeled by including labeled nucleotides in the amplification reactions.

Nucleic acid molecules corresponding to one or more tag SNPs or haplotype blocks including the tag SNP can also be detected by hybridization procedures using a labeled nucleic acid probe, such as a probe that detects only one alternative allele at a marker locus. Most commonly, the target nucleic acid (or amplified target nucleic acid) is separated based on size or charge and transferred to a solid support. The solid support (such as membrane made of nylon or nitrocellulose) is contacted with a labeled nucleic acid probe, which hybridizes to it complementary target under suitable hybridization conditions to form a hybridization complex.

Hybridization conditions for a given combination of array and target material can be optimized routinely in an empirical manner close to the Tₘ of the expected duplexes, thereby maximizing the discriminating power of the method. For example, the hybridization conditions can be selected to permit discrimination between matched and mismatched oligonucleotides. Hybridization conditions can be chosen to correspond to those known to be suitable in standard procedures for hybridization to filters (and optionally for hybridization to arrays). In particular, temperature is controlled to substantially eliminate formation of duplexes between sequences other than an exactly complementary allele of the selected marker. A variety of known hybridization solvents can be employed, the choice being dependent on considerations known to one of skill in the art (see U.S. Pat. No. 5,981,185).

Once the target nucleic acid molecules have been hybridized with the labeled probes, the presence of the hybridization complex can be analyzed, for example by detecting the complexes.

Methods for detecting hybridized nucleic acid complexes are well known in the art. In one example, detection includes detecting one or more labels present on the oligonucleotides, the target (e.g., amplified) sequences, or both. Detection can include treating the hybridized complex with a buffer and/or a conjugating solution to effect conjugation or coupling of the hybridized complex with the detection label, and treating the conjugated, hybridized complex with a detection reagent. In one example, the conjugating solution includes streptavidin alkaline phosphatase, avidin alkaline phosphatase, or horseradish peroxidase. Specific, non-limiting examples of conjugating solutions include streptavidin alkaline phosphatase, avidin alkaline phosphatase, or horseradish peroxidase. The conjugated, hybridized complex can be treated with a detection reagent. In one example, the detection reagent includes enzyme-labeled fluorescence reagents or calorimetric reagents. In one specific non-limiting example, the detection reagent is enzyme-labeled fluorescence reagent (ELF) from Molecular Probes, Inc. (Eugene, Oreg.). The hybridized complex can then be placed on a detection device, such as an ultraviolet (UV) transilluminator (manufactured by UVP, Inc. of Upland, Calif.). The signal is developed and the increased signal intensity can be recorded with a recording device, such as a charge coupled device (CCD) camera (manufactured by Photometrics, Inc. of Tucson, Ariz.). In particular examples, these steps are not performed when radiolabels are used. In particular examples, the method further includes quantification, for instance by determining the amount of hybridization.

Allele Specific PCR:

Allele-specific PCR differentiates between target regions differing in the presence of absence of a variation or polymorphism. PCR amplification primers are chosen based upon their complementarity to the target sequence, such as nucleic acid sequence in including a SNP, a specified region of an allele including an SNP, or to the SNP itself. The primers bind only to certain alleles of the target sequence. This method is described by Gibbs, Nucleic Acid Res. 17:12427 2448, 1989, herein incorporated by reference. In some examples, a commercially available assay based upon allele-specific PCR SNP detection chemistry is utilized, such as a SNPtype™ Assay, Dynamic Array IFCs or other SNP genotyping product from FLUIDIGM® is utilized.

Allele Specific Oligonucleotide Screening Methods:

Further screening methods employ the allele-specific oligonucleotide (ASO) screening methods (e.g. see Saiki et al., Nature 324:163-166, 1986). Oligonucleotides with one or more base pair mismatches are generated for any particular allele or haplotype block. ASO screening methods detect mismatches between one allele (or haplotype block) in the target genomic or PCR amplified DNA and the other allele (or haplotype block), showing decreased binding of the oligonucleotide relative to the second allele (i.e. the other allele) oligonucleotide. Oligonucleotide probes can be designed that under low stringency will bind to both polymorphic forms of the allele, but which at high stringency, only bind to the allele to which they correspond. Alternatively, stringency conditions can be devised in which an essentially binary response is obtained, i.e., an ASO corresponding to a variant form of the target gene will hybridize to that allele (haplotype block), and not to the reference allele (haplotype block).

Ligase Mediated Allele Detection Method:

Ligase can also be used to detect point mutations in a ligation amplification reaction (e.g. as described in Wu et al., Genomics 4:560-569, 1989). The ligation amplification reaction (LAR) utilizes amplification of specific DNA sequence using sequential rounds of template dependent ligation (e.g. as described in Wu, supra, and Barany, Proc. Nat. Acad. Sci. 88:189-193, 1990).

Denaturing Gradient Gel Electrophoresis:

Amplification products generated using the polymerase chain reaction can be analyzed by the use of denaturing gradient gel electrophoresis. Different alleles (haplotype blocks) can be identified based on the different sequence-dependent melting properties and electrophoretic migration of DNA in solution. DNA molecules melt in segments, termed melting domains, under conditions of increased temperature or denaturation. Each melting domain melts cooperatively at a distinct, base-specific melting temperature (T_{M}). Melting domains are at least 20 base pairs in length, and can be up to several hundred base pairs in length.

Differentiation between alleles (haplotype blocks) based on sequence specific melting domain differences can be assessed using polyacrylamide gel electrophoresis, as described in Chapter 7 of Erlich, ed., PCR Technology, Principles and Applications for DNA Amplification, W. H. Freeman and Co., New York (1992).

Generally, a target region to be analyzed by denaturing gradient gel electrophoresis is amplified using PCR primers flanking the target region. The amplified PCR product is applied to a polyacrylamide gel with a linear denaturing gradient as described in Myers et al., Meth. Enzymol. 155:501-527, 1986, and Myers et al., in Genomic Analysis, A Practical Approach, K. Davies Ed. IRL Press Limited, Oxford, pp. 95 139, 1988. The electrophoresis system is maintained at a temperature slightly below the Tm of the melting domains of the target sequences.

In an alternative method of denaturing gradient gel electrophoresis, the target sequences can be initially attached to a stretch of GC nucleotides, termed a GC clamp, as described in Chapter 7 of Erlich, supra. In one example, at least 80% of the nucleotides in the GC clamp are either guanine or cytosine. In another example, the GC clamp is at least 30 bases long. This method is particularly suited to target sequences with high Tₘ's.

Generally, the target region is amplified by polymerase chain reaction. One of the oligonucleotide PCR primers carries at its 5′ end, the GC clamp region, at least 30 bases of the GC rich sequence, which is incorporated into the 5′ end of the target region during amplification. The resulting amplified target region is run on an electrophoresis gel under denaturing gradient conditions. DNA fragments differing by a single base change will migrate through the gel to different positions, which can be visualized by ethidium bromide staining.

Temperature Gradient Gel Electrophoresis:

Temperature gradient gel electrophoresis (TGGE) is based on the same underlying principles as denaturing gradient gel electrophoresis, except the denaturing gradient is produced by differences in temperature instead of differences in the concentration of a chemical denaturant. Standard TGGE utilizes an electrophoresis apparatus with a temperature gradient running along the electrophoresis path. As samples migrate through a gel with a uniform concentration of a chemical denaturant, they encounter increasing temperatures. An alternative method of TGGE, temporal temperature gradient gel electrophoresis (TTGE or tTGGE) uses a steadily increasing temperature of the entire electrophoresis gel to achieve the same result. As the samples migrate through the gel the temperature of the entire gel increases, leading the samples to encounter increasing temperature as they migrate through the gel. Preparation of samples, including PCR amplification with incorporation of a GC clamp, and visualization of products are the same as for denaturing gradient gel electrophoresis.

Single-Strand Conformation Polymorphism Analysis:

Target sequences, such as alleles or haplotype blocks can be differentiated using single-strand conformation polymorphism analysis, which identifies base differences by alteration in electrophoretic migration of single stranded PCR products, for example as described in Orita et al., Proc. Nat. Acad. Sci. 85:2766-2770, 1989. Amplified PCR products can be generated as described above, and heated or otherwise denatured, to form single stranded amplification products. Single-stranded nucleic acids can refold or form secondary structures which are partially dependent on the base sequence. Thus, electrophoretic mobility of single-stranded amplification products can detect base-sequence difference between alleles or haplotype blocks.

Chemical or Enzymatic Cleavage of Mismatches:

Differences between target sequences, such as alleles or haplotype blocks, can also be detected by differential chemical cleavage of mismatched base pairs, for example as described in Grompe et al., Am. J. Hum. Genet. 48:212-222, 1991. In another method, differences between target sequences, such as alleles or haplotype blocks, can be detected by enzymatic cleavage of mismatched base pairs, as described in Nelson et al., Nature Genetics 4:11-18, 1993. Briefly, genetic material from an animal and an affected family member can be used to generate mismatch free heterohybrid DNA duplexes. As used herein, “heterohybrid” means a DNA duplex strand comprising one strand of DNA from one animal, and a second DNA strand from another animal, usually an animal differing in the phenotype for the trait of interest. Positive selection for heterohybrids free of mismatches allows determination of small insertions, deletions or other polymorphisms.

Non-Gel Systems:

Other possible techniques include non-gel systems such as TaqMan™ (Perkin Elmer). In this system oligonucleotide PCR primers are designed that flank the mutation in question and allow PCR amplification of the region. A third oligonucleotide probe is then designed to hybridize to the region containing the base subject to change between different alleles of the gene. This probe is labeled with fluorescent dyes at both the 5′ and 3′ ends. These dyes are chosen such that while in this proximity to each other the fluorescence of one of them is quenched by the other and cannot be detected. Extension by Taq DNA polymerase from the PCR primer positioned 5′ on the template relative to the probe leads to the cleavage of the dye attached to the 5′ end of the annealed probe through the 5′ nuclease activity of the Taq DNA polymerase. This removes the quenching effect allowing detection of the fluorescence from the dye at the 3′ end of the probe. The discrimination between different DNA sequences arises through the fact that if the hybridization of the probe to the template molecule is not complete (there is a mismatch of some form) the cleavage of the dye does not take place. Thus only if the nucleotide sequence of the oligonucleotide probe is completely complimentary to the template molecule to which it is bound will quenching be removed. A reaction mix can contain two different probe sequences each designed against different alleles that might be present thus allowing the detection of both alleles in one reaction.

Non-PCR Based Allele Detection:

The identification of a DNA sequence can be made without an amplification step, based on polymorphisms including restriction fragment length polymorphisms in a subject and a control, such as a family member. Hybridization probes are generally oligonucleotides which bind through complementary base pairing to all or part of a target nucleic acid. Probes typically bind target sequences lacking complete complementarity with the probe sequence depending on the stringency of the hybridization conditions. The probes can be labeled directly or indirectly, such that by assaying for the presence or absence of the probe, one can detect the presence or absence of the target sequence. Direct labeling methods include radioisotope labeling, such as with ³²P or ³⁵S. Indirect labeling methods include fluorescent tags, biotin complexes which can be bound to avidin or streptavidin, or peptide or protein tags. Visual detection methods include photoluminescents, Texas red, rhodamine and its derivatives, red leuco dye and 3,3′,5,5′-tetramethylbenzidine (TMB), fluorescein, and its derivatives, dansyl, umbelliferone and the like or with horse radish peroxidase, alkaline phosphatase and the like.

Hybridization probes include any nucleotide sequence capable of hybridizing to a nucleic acid sequence wherein a polymorphism is present that is associated with a particular skin factor, and thus defining a genetic marker, including a restriction fragment length polymorphism, a hypervariable region, repetitive element, or a variable number tandem repeat. Hybridization probes can be any gene or a suitable analog. Further suitable hybridization probes include exon fragments or portions of cDNAs or genes known to map to the relevant region of the chromosome.

Exemplary tandem repeat hybridization probes for use in the methods disclosed are those that recognize a small number of fragments at a specific locus at high stringency hybridization conditions, or that recognize a larger number of fragments at that locus when the stringency conditions are lowered.

Arrays for Detecting Nucleic Acid:

In particular examples, the methods can be performed using an array that includes a plurality of markers. Such arrays can include nucleic acid molecules. In one example, the array includes nucleic acid oligonucleotide probes that can hybridize to one or more alleles.

Arrays can be used to detect the presence of amplified sequences including one or more SNPs of interest using specific oligonucleotide probes. Additionally, if an internal control nucleic acid sequence was amplified in the amplification reaction, an oligonucleotide probe can be included to detect the presence of this amplified nucleic acid molecule. The oligonucleotide probes bound to the array can specifically bind sequences amplified in the amplification reaction (such as under high stringency conditions).

The methods and apparatus in accordance with the present disclosure takes advantage of the fact that under appropriate conditions oligonucleotides form base-paired duplexes with nucleic acid molecules that have a complementary base sequence. The stability of the duplex is dependent on a number of factors, including the length of the oligonucleotides, the base composition, and the composition of the solution in which hybridization is performed. The effects of base composition on duplex stability can be reduced by carrying out the hybridization in particular solutions, for example in the presence of high concentrations of tertiary or quaternary amines.

The thermal stability of the duplex is also dependent on the degree of sequence similarity between the sequences. By carrying out the hybridization at temperatures close to the anticipated Tₘ's of the type of duplexes expected to be formed between the target sequences and the oligonucleotides bound to the array, the rate of formation of mis-matched duplexes can be substantially reduced.

The length of each oligonucleotide sequence employed in the array can be selected to optimize binding to a specific allele of a marker locus associated with a particular skin factor. An optimum length for use with a particular marker nucleic acid sequence under specific screening conditions can be determined empirically. Thus, the length for each individual element of the set of oligonucleotide sequences included in the array can be optimized for screening. In one example, oligonucleotide probes are from about 20 to about 35 nucleotides in length or about 25 to about 40 nucleotides in length.

The oligonucleotide probe sequences forming the array can be directly linked to the support, for example via the 5′- or 3′-end of the probe. In one example, the oligonucleotides are bound to the solid support by the 5′ end. However, one of skill in the art can determine whether the use of the 3′ end or the 5′ end of the oligonucleotide is suitable for bonding to the solid support. In general, the internal complementarity of an oligonucleotide probe in the region of the 3′ end and the 5′ end determines binding to the support. Alternatively, the oligonucleotide probes can be attached to the support by sequences such as oligonucleotides or other molecules that serve as spacers or linkers to the solid support.

In particular examples, the array is a microarray formed from glass (silicon dioxide). Suitable silicon dioxide types for the solid support include, but are not limited to: aluminosilicate, borosilicate, silica, soda lime, zinc titania and fused silica (for example see Schena, Micraoarray Analysis. John Wiley & Sons, Inc, Hoboken, N. J., 2003). The attachment of nucleic acids to the surface of the glass can be achieved by methods known in the art, for example by surface treatments that form from an organic polymer. Particular examples include, but are not limited to: polypropylene, polyethylene, polybutylene, polyisobutylene, polybutadiene, polyisoprene, polyvinylpyrrolidine, polytetrafluroethylene, polyvinylidene difluroide, polyfluoroethylene-propylene, polyethylenevinyl alcohol, polymethylpentene, polycholorotrifluoroethylene, polysulfornes, hydroxylated biaxially oriented polypropylene, aminated biaxially oriented polypropylene, thiolated biaxially oriented polypropylene, etyleneacrylic acid, thylene methacrylic acid, and blends of copolymers thereof (see U.S. Pat. No. 5,985,567), organosilane compounds that provide chemically active amine or aldehyde groups, epoxy or polylysine treatment of the microarray. Another example of a solid support surface is polypropylene.

In general, suitable characteristics of the material that can be used to form the solid support surface include: being amenable to surface activation such that upon activation, the surface of the support is capable of covalently attaching a biomolecule such as an oligonucleotide thereto; amenability to “in situ” synthesis of biomolecules; being chemically inert such that at the areas on the support not occupied by the oligonucleotides are not amenable to non-specific binding, or when non-specific binding occurs, such materials can be readily removed from the surface without removing the oligonucleotides.

In one example, the surface treatment is amine-containing silane derivatives. Attachment of nucleic acids to an amine surface occurs via interactions between negatively charged phosphate groups on the DNA backbone and positively charged amino groups (Schena, Micraoarray Analysis. John Wiley & Sons, Inc, Hoboken, N. J., 2003). In another example, reactive aldehyde groups are used as surface treatment. Attachment to the aldehyde surface is achieved by the addition of 5′-amine group or amino linker to the DNA of interest. Binding occurs when the nonbonding electron pair on the amine linker acts as a nucleophile that attacks the electropositive carbon atom of the aldehyde group.

A wide variety of array formats can be employed in accordance with the present disclosure. One example includes a linear array of oligonucleotide bands, generally referred to in the art as a dipstick. Another suitable format includes a two-dimensional pattern of discrete cells (such as 4096 squares in a 64 by 64 array). As is appreciated by those skilled in the art, other array formats including, but not limited to slot (rectangular) and circular arrays are equally suitable for use (see U.S. Pat. No. 5,981,185). In one example, the array is formed on a polymer medium, which is a thread, membrane or film. An example of an organic polymer medium is a polypropylene sheet having a thickness on the order of about 1 mil. (0.001 inch) to about 20 mil., although the thickness of the film is not critical and can be varied over a fairly broad range. Biaxially oriented polypropylene (BOPP) films are also suitable in this regard; in addition to their durability, BOPP films exhibit a low background fluorescence. In a particular example, the array is a solid phase, Allele-Specific Oligonucleotides (ASO) based nucleic acid array.

The array formats of the present disclosure can be included in a variety of different types of formats. A “format” includes any format to which the solid support can be affixed, such as microtiter plates, test tubes, inorganic sheets, dipsticks, and the like. For example, when the solid support is a polypropylene thread, one or more polypropylene threads can be affixed to a plastic dipstick-type device; polypropylene membranes can be affixed to glass slides. The particular format is, in and of itself, unimportant. All that is necessary is that the solid support can be affixed thereto without affecting the functional behavior of the solid support or any biopolymer absorbed thereon, and that the format (such as the dipstick or slide) is stable to any materials into which the device is introduced (such as clinical samples and hybridization solutions).

The arrays of the present disclosure can be prepared by a variety of approaches. In one example, oligonucleotide or protein sequences are synthesized separately and then attached to a solid support (see U.S. Pat. No. 6,013,789). In another example, sequences are synthesized directly onto the support to provide the desired array (see U.S. Pat. No. 5,554,501). Suitable methods for covalently coupling oligonucleotides and proteins to a solid support and for directly synthesizing the oligonucleotides or proteins onto the support are known to those working in the field; a summary of suitable methods can be found in Matson et al., Anal. Biochem. 217:306-10, 1994. In one example, the oligonucleotides are synthesized onto the support using conventional chemical techniques for preparing oligonucleotides on solid supports (such as see PCT Publication No. WO 85/01051 and PCT Publication No. WO 89/10977, or U.S. Pat. No. 5,554,501).

A suitable array can be produced using automated means to synthesize oligonucleotides in the cells of the array by laying down the precursors for the four bases in a predetermined pattern. Briefly, a multiple-channel automated chemical delivery system is employed to create oligonucleotide probe populations in parallel rows (corresponding in number to the number of channels in the delivery system) across the substrate. Following completion of oligonucleotide synthesis in a first direction, the substrate can then be rotated by 90° to permit synthesis to proceed within a second (2°) set of rows that are now perpendicular to the first set. This process creates a multiple-channel array whose intersection generates a plurality of discrete cells.

In particular examples, the oligonucleotide probes on the array include one or more labels, which permit detection of oligonucleotide probe:target sequence hybridization complexes.

Without further elaboration, it is believed that one skilled in the art can, using this description, utilize the present disclosure to its fullest extent. The following example is illustrative only, and not limiting of the remainder of the disclosure in any way whatsoever.

### EXAMPLES

### Example 1

This example provides the results from a clinical study of several hundred individuals illustrating the effectiveness of the presently disclosed method and protocols. In particular, this example validates SNP: MMP-1/Collagen Formation 11q21q22 for use in disclosed methods and protocols.

The timely breakdown of extracellular matrix (ECM) is essential for embryonic development, morphogenesis, reproduction, and tissue resorption and remodeling. The matrix metalloproteinases (MMPs), also called matrixins, are thought to play a central role in these processes. The expression of most matrixins is transcriptionally regulated by growth factors, hormones, cytokines, and cellular transformation. The proteolytic activities of MMPs are precisely controlled during activation from their precursors and inhibition by endogenous inhibitors, α-macroglobulins, and tissue inhibitors of metalloproteinases (TIMPs). MMPs are also involved in wound healing, a tissue-remodeling process which involves the migration of keratinocytes at the edge of the wound to re-epithelialize the damaged surface.

Damage to human skin due to ultraviolet light from the sun (photoaging) and damage occurring as a consequence of the passage of time (chronologic or natural aging) are considered to be distinct entities. Photoaging is caused in part by damage to skin connective tissue by increased elaboration of collagen-degrading matrix metalloproteinases, and by reduced collagen synthesis. As matrix metalloproteinase levels are known to rise in fibroblasts as a function of age, and as oxidant stress is believed to underlie changes associated with both photoaging and natural aging, it was determined whether natural skin aging, like photoaging, gives rise to increased matrix metalloproteinases and reduced collagen synthesis.

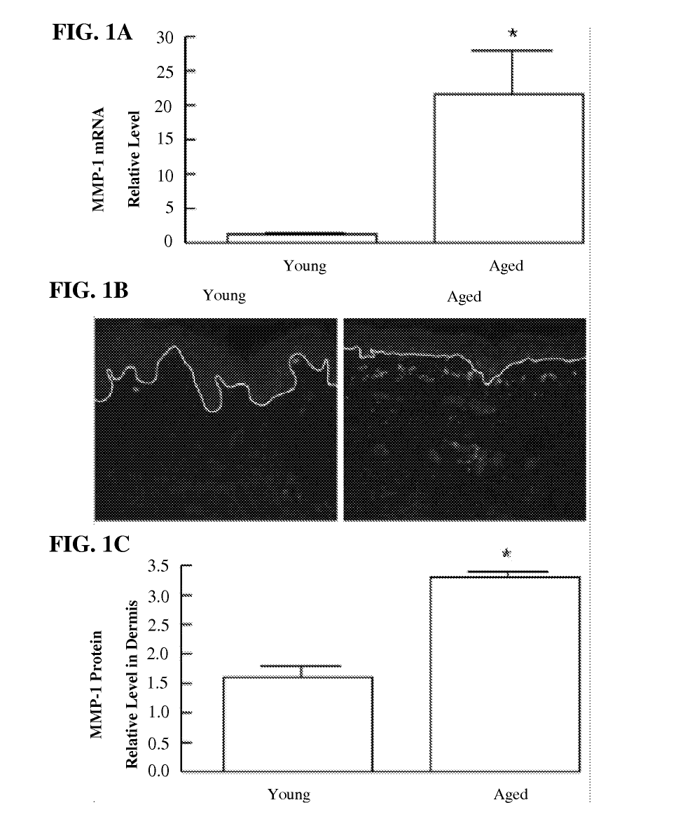

In human skin, degradation of mature collagen fibrils can be initiated by matrix metalloproteinase-1 (MMP-1). MMP-1 is also known as interstitial collagenase and fibroblast collagenase. Oxidative stress, shown above to be increased in aged dermis, has been shown to increase MMP-1 expression in cultured cell models. Therefore, the dermis was dissected from whole skin and measured MMP-1 mRNA levels. As shown in FIG. 1A, MMP-1 mRNA was increased eightfold in aged, compared with young dermis. Consistent with these data, MMP-1 protein levels, detected by immunofluorescence, were significantly elevated in aged, compared with young human dermis (FIG. 1B). Increased MMP-1 expression in aged human skin was predominantly localized to fibroblasts in the upper dermis, where fibroblasts display increased levels of oxidants. Quantitation of immunostaining indicated twofold increase in MMP-1 protein in aged, compared with young dermis (FIG. 1B). In addition, aged skin exhibited higher levels of collagenase activity as revealed by in situ zymography. This elevated activity, revealed as darkened areas resulting from hydrolysis of FITC-labeled collagen, was localized to the upper dermis, consistent with localization of MMP-1 protein (FIG. 1C).

Based upon the findings, the following therapies were recommended and/or provided:
1 Antioxidant therapy, including recommending Vitamins C and E supplements to increase the collagen content and reduce MMP-1.
2 Determining if high cholesterol (which can decrease collagen) is a cause of the increased MMP1 expression. If so, recommend a dietary regimen to reduce high cholesterol.
3 Reduce or limit exposure to UV light. UVB damage to keratinocytes of the epidermis may participate in the destruction of collagen in the dermis by release of soluble mediators the signal fibroblasts to release MMP-1.
4 Provide DNA repair enzymes support in topical products and nutritional products so as to reduce MMP-1 induction.
5 Soy isoflavones suppress MMP production, and therefore may be provided.
6 Recommend topical or oral glutathione or glutathione enhancer/supporters so as to minimize the effects of oxidative stress as it helps to increase intracellular glutathione and accelerate wound healing by increasing the contraction capacity of fibroblasts and preventing keratinocytes from apoptosis.
7 Recommend topical DHEA or oral to inhibit UV induced MMP-1 production and the UV induced decreased of procollagen synthesis.
8 Provide Coenzyme Q10 to suppresses the UV radiation or interleukin 6-induced inflammatory response in dermal fibroblasts, to block the UVR induction of the matrix-eroding enzyme, MMP-1, and suppresses inflammation

### Example 2

This example provides a method for generating a personalized skincare profile and providing skincare and dietary regimen based upon the personalized skincare profile.

### Identification of SNPs

SNPs were identified through a search of SNPedia, the NCBI Variation Database (dsSNP), the HapMap Database as well as other scientific databases of SNP's and/or genetic markers to identify key factors/words and phrases that would reveal scientific information pertaining to biochemical factors that can affect or influence the health and beauty of the skin. The resulting data and literature created from this search process was then reviewed to determine whether the biological significance of the identified polymorphisms or genetic markers was supported by scientific resources such as human studies and/or published peer-reviewed medical journals.

Criteria for the significance and scientific validity of SNPs included measuring their minor allele frequencies (MAFs, see below) which were verified in the dsSNP database and/or SNPedia, peer reviewed literature, or published human studies, and this information was used as criteria for inclusion or exclusion of the most relevant skin related SNP's in the panel/profile. A PubMed search was carried out for each polymorphism and the frequency was determined. The resulting literature covered under each SNP was reviewed to determine the significance of the polymorphism supported by human studies, and/or it was verified in multiple human studies, and/or the results were published in peer-reviewed journals. Any SNP that was associated with at least one or more of the five areas relevant to skin health and beauty was given consideration.

A process to cross-check the relevance, validity and significance of SNPs was used and included such things as their evaluation through the Genome Wide Association Studies (GWAS). After compiling a list of the most relevant skin health and beauty polymorphisms, the SNPs were grouped into one or more of the following categories:
1. Collagen production
2. Sun protection
3. Antioxidant production
4. Glycation
5. Inflammation

A process to prioritize SNPs in order of highest to lowest significance and relevance to skin health and beauty was done based on the quality and strength of the scientific data and relevance to their grouping. Abstracts, published peer-reviewed papers and other available literature were compiled as support material for the process and research carried out.

### Methodology for Maintaining Sample Integrity

To maintain sample integrity when analyzing the DNA, SNPs relevant to skin health and beauty for the disclosed system, a panel of one or more SNPs, possibly including a sex marker, were compiled that provide a DNA fingerprint for each individual. This process included methods of Cross et al (2009. Development of a fingerprinting panel using medically relevant polymorphisms. BMC Med Gen. 2:17, which is hereby incorporated by reference in its entirety). The criteria that were adapted from this method for the disclosed test included the following:
1. The polymorphisms may have a confirmed minor allele frequency (MAF) of 0.20. This methodology speaks to their significance and relevance in human populations.
2. The polymorphisms must be relevant in one or more areas of skin health and beauty.
3. They must be easily detectable (Sequenom or other similar platform).

### Choosing a Lab for DNA Testing

After the SNP panel was finalized, a search was conducted to identify a lab to test the DNA samples. The lab obtained reagents such as primers from commercial resources such as Life Technologies that were capable of identifying the desired SNPs in human genomic DNA followed by confirmation that the SNPs in the panel were accurately verified by the testing method and reagents used.

### DNA Test Kit

The SNP panel was finalized, a lab was chosen to carry out the DNA analyses, and a test kit to obtain the DNA samples was designed that included:
1. A swab for collecting the DNA sample from the inside of the cheek.
2. A questionnaire to be filled out by the individual.
3. An envelope into which the DNA swab is placed for mailing or delivering.
4. Instructions for collecting and submitting the sample.
5. A mailing envelope

### DNA Testing

The DNA testing process included the following:
1. An subject obtained the test by either purchasing online or a storefront (such as a healthcare provider or commercial entity that sells nutritional supplements).
2. The subject completed the questionnaire included in the DNA test kit to the best of their ability.
3. The subject removed the protected DNA test swab from a sealed wrapper and rubbed the swab across the inside of their cheek to obtain the DNA sample. They then closed the slider on the swab, filled out the information on the swab as per the instructions, placed it in the envelope provided, and filled out the questionnaire as per the instructions.
4. The individual provided the DNA sample (such as by mail or hand delivery) to SKINSHIFT®, a skincare company.
5. SKINSHIFT® forwarded the sample to the DNA testing lab.
6. The lab isolated the DNA and performs the testing.
7. A computer program was developed to weigh the statistical value of the DNA test results to create a report for the subject (as described in detail below).
8. The data was compiled and data/results were formatted into a DNA report.

### The DNA Report

The DNA report was statistically tabulated and electronically created through a computer system, and was comprised of the following:
1. A summary page that lists the overall risk ratings of high, medium, or low under each of up to 5 or more categories of skin health and beauty.
2. A page describing the biological effects and the visible signs of each of the up to or more than 5 categories of skin health and beauty.
3. A page for each of the up to or more than 5 categories describing the category in greater detail and listing the marker-specific results for the individual is created. A rating system included the following:
   i. A value of 0.25 was assigned to a result of homozygous for the non-ideal allele at the at risk SNP location (non-ideal function), which indicated high risk and was represented by a deficient rating;
   ii. A value of 0.50 was assigned to a result of heterozygous for the non-ideal allele at the risk SNP location, which indicated medium risk and was represented by a sub-normal rating; and
   iii. A value of 1.00 was assigned to a result of homozygous for the ideal allele at the risk SNP location, which indicated low risk and was represented by a normal rating.
4. A computer program/algorithm calculated a numerical value for each category of skin health and beauty that was based on the average of the numbers provided for each SNP contained in a skin health category and generates an overall numerical rating.
5. High risk was considered an average numerical value of 0.25-0.5. Medium risk was considered to be a numerical average of 0.5-0.75 and low risk was considered to be a numerical value of 0.75-1.0.
6. Marker specific results were listed by SNP and included:
   The affected gene or marker
   The chromosomal location
   The ideal genotype
   The actual genotype
   The rating
7. Specific product recommendations for each individual were created according to a program based on each individual's report. Foundational products were recommended for everyone and these did not represent the unique product recommendations made for the individual based on their genetic results. These products are relevant; however, in that they set the stage for optimal results for personalized serums and supplements, based on each individual's unique DNA report. These foundational products may have included:
   d. An Antioxidant Cleanser
   e. A Balancing Toner
   f. An Antioxidant Moisturizer

Following recommendations for non-customized products, one or more custom-designed products were recommended based on the DNA report. These recommendations included a combination of treatment serums/nutritional products that address the individual strengths and weaknesses of the individuals skin as determined by their SKINSHIFT report.

These customized recommendations included products such as:

Serums:
1. Vitamin C Treatment Serum
2. Hyaluronic Moisture Treatment Serum
3. Wrinkle Treatment Serum
4. Calming Treatment Serum

Supplements:
5. Antioxidant Defense
6. Glycation Defense
7. Sun Defense
8. Collagen Defense
9. Inflammation Defense
   The determination as to which products would be custom recommended was based on an algorithmic protocol that matched the ingredients in the products to the weakness that the person's DNA analysis revealed as identified in their medium or high risk categories. This was programmed based on an algorithm into the computer system that generates the DNA report. High risk categories were the most important category recommendations to follow as they provided the individual the greatest opportunity to improve the health and appearance of their skin followed by the medium risk category recommendations. For instance, if a person was determined to be medium or high risk for collagen, products were recommended that supported healthy collagen production and maintenance in the skin. The ingredients in the products that support healthy collagen production and maintenance were selected and put into the skin care products and nutritional supplements based on clinical research that supported the clinical effectiveness of the ingredients included in the product on the category to which it applies. Product recommendations were organized into high risk and medium risk categories on the report for ease of understanding and product selection by the individual. If a product was already listed under a high-risk category, it was not be relisted under a medium risk category as it was already selected by the algorithm and recommended.

Following the formulation process and algorithmic recommendations above, products were clinically used and evaluated in the inventor's clinical practice to ensure that the effectiveness, aesthetic appeal and client satisfaction are of the highest standards possible.

After reviewing the DNA report, the report was sent to the subject and an optional customer service meeting/call was set up for those who desired to go over the results and product recommendations, and/or had questions regarding their report with a SKINSHIFT® representative.

Analysis of the data from this study confirmed the ability of the laboratory to accurately and reproducibly identify selected SNPs and verify their frequency in the population studied. Population frequencies were compared to the medical literature for accuracy. An algorithm was created to mathematically calculate the impact of each SNP on the applicable function. The example demonstrates that the disclosed methods provide clients/patients a highly personalized method by which they can objectively identify biological strengths and weaknesses in their genetic coding that affected the health and beauty of their skin. Further, the disclosed topical and nutritional protocols demonstrate that the methods beneficially influence the targeted skincare area.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. I therefore claim as my invention all that comes within the scope and spirit of these claims.

TABLE 1
RS numbers for SNPs associated with one or more
areas of skin health

| | | |
|---|---|---|
| rs149392574 | rs2854510 | rs6591255 |
| rs144780150 | rs2682586 | rs4986949 |
| rs17884110 | rs2682585 | rs4147581 |
| rs17881293 | rs2682558 | rs2370143 |
| rs17878931 | rs2307187 | rs1871042 |
| rs11292517 | rs2307184 | rs1871041 |
| rs7945189 | rs2307177 | rs1138272 |
| rs7125320 | rs2293036 | rs947895 |
| rs7125062 | rs1799782 | rs762803 |
| rs5031036 | rs1799780 | rs749174 |
| rs3213460 | rs1001581 | rs4891 |
| rs2239008 | rs939461 | rs1695 |
| rs2075847 | rs915927 | rs281865510 |
| rs2071232 | rs25496 | rs281865510 |
| rs2071231 | rs25491 | rs281865509 |
| rs2071230 | rs25489 | rs281865508 |
| rs1938901 | rs25487 | rs281865478 |
| rs1799750 | rs25486 | rs63749957 |
| rs1144393 | rs25478 | rs281865475 |
| rs996999 | rs3547 | rs281865474 |
| rs470558 | rs3218536 | rs281865463 |
| rs470132 | rs3218501 | rs281865462 |
| rs5854 | rs3218499 | rs281865461 |
| rs104886287 | rs3218493 | rs281865460 |
| rs104886229 | rs3218472 | rs281865161 |
| rs121908252 | rs3218461 | rs281865160 |
| rs80338741 | rs3218455 | rs281865159 |
| rs5030737 | rs3218454 | rs281865158 |
| rs1800450 | rs3218425 | rs281865157 |
| rs111033548 | rs3218418 | rs281865154 |
| rs111033545 | rs3218417 | rs281865153 |
| rs74821926 | rs3218410 | rs281865142 |
| rs121912833 | rs3218408 | rs41297589 |
| rs121912829 | rs3218402 | rs4645843 |
| rs1800255 | rs3218400 | rs3093668 |
| rs8179164 | rs3218395 | rs3093665 |
| rs3811699 | rs3218385 | rs3093664 |
| rs1800668 | rs3218384 | rs3093662 |
| rs1050450 | rs3218374 | rs3093661 |
| rs3448 | rs3218373 | rs1800750 |
| rs4902346 | rs17884057 | rs1800630 |
| rs2071566 | rs17881180 | rs1800629 |
| rs142544404 | rs10432782 | rs1800610 |
| rs34499590 | rs7277748 | rs1799964 |
| rs17412175 | rs4998557 | rs1799724 |
| rs17097182 | rs4816407 | rs361525 |
| rs13306526 | rs2234694 | rs673 |
| rs13306523 | rs2070424 | rs281865510 |
| rs12409877 | rs1041740 | rs281865510 |
| rs12405556 | rs5746151 | rs281865509 |
| rs12067936 | rs5746136 | rs281865508 |
| rs12065099 | rs5746134 | rs281865478 |
| rs10889557 | rs5746129 | rs63749957 |
| rs10889553 | rs5746105 | rs281865475 |
| rs10749753 | rs5746097 | rs281865474 |
| rs10493380 | rs5746096 | rs281865463 |
| rs10493379 | rs5746092 | rs281865462 |
| rs9436748 | rs4987023 | rs281865461 |
| rs9436746 | rs2855116 | rs281865460 |
| rs9436740 | rs2842958 | rs281865161 |
| rs1805094 | rs2758346 | rs281865160 |
| rs6700896 | rs2758330 | rs281865159 |
| rs1137100 | rs10370 | rs281865158 |
| rs6059743 | rs8031 | rs281865157 |
| rs6058017 | rs4880 | rs281865154 |
| rs1129414 | rs17881426 | rs281865153 |
| rs819162 | rs17880362 | Rs1001179 |
| rs819136 | rs17878863 | Rs1050450 |
| rs819135 | rs13306703 | Rs1695 |
| rs12218196 | rs8192291 | Rs2917666 |
| rs10827337 | rs8192290 | Rs4880 |
| rs10763976 | rs8192288 | Rs1062470 |
| rs4379776 | rs8192287 | Rs4688761 |
| rs3851068 | rs2855262 | Rs1309582 |
| rs3781128 | rs2695232 | Rs16845861 |
| rs2496720 | rs2695231 | Rs10212372 |
| rs2384137 | rs2536512 | Rs4688761 |
| rs2252655 | rs2536511 | Rs9883988 |
| rs2025459 | rs1799895 | Rs10934938 |
| rs2002773 | rs1007991 | Rs9399005 |
| rs1753586 | rs800444 | Rs1800449 |
| rs1657224 | rs699474 | Rs3925942 |
| rs1359281 | rs699473 | Rs255690 |
| rs942431 | rs72549341 | Rs154001 |
| rs773991 | rs34143170 | Rs8032158 |
| rs773985 | rs3766934 | Rs2070600 |
| rs678188 | rs3753661 | Rs2736654 |
| rs673009 | rs3753660 | Rs4112788 |
| rs657477 | rs3753658 | Rs1137101 |
| rs650212 | rs3738047 | rs743409 |
| rs647758 | rs3738043 | rs17561 |
| rs626859 | rs3738042 | rs11568821 |
| rs626394 | rs2854461 | rs1800470 |
| rs563507 | rs2854456 | rs1800471 |
| rs493392 | rs2854455 | rs4911414 |
| rs3916876 | rs2854450 | rs1052133 |
| rs3916874 | rs2740171 | rs1805008 |
| rs3916840 | rs2740170 | rs25487 |
| rs3810366 | rs2740168 | rs1408799 |
| rs1799793 | rs2671272 | rs1393350 |
| rs1799792 | rs2292568 | rs1042602 |
| rs1799791 | rs2260863 | rs1126809 |
| rs1799787 | rs41507953 | rs1393350 |
| rs1052555 | rs7837347 | rs1408799 |
| rs238416 | rs7003694 | rs1800401 |
| rs238415 | rs4149244 | rs1800407 |
| rs238406 | rs2071575 | rs13289 |
| rs238405 | rs1126452 | rs26722 |
| rs238403 | rs1042064 | rs16891982 |
| rs171140 | rs1042032 | rs17426596 |
| rs50872 | rs751141 | rs1042571 |
| rs50871 | rs747276 | rs4911414 |
| rs13181 | rs721619 | rs1015362 |
| rs34612342 | rs16980438 | rs6058017 |
| rs17050550 | rs757291 | rs17782078 |
| rs3219008 | rs45549733 | rs3886999 |
| rs2072668 | rs45506591 | rs1048661 |
| rs1805373 | rs36211089 | rs3825942 |
| rs1052133 | rs36211088 | rs35391 |
| rs293795 | rs36211087 | rs28777 |
| rs293794 | rs17593068 | rs1689182 |
| rs159153 | rs11553890 | rs426654 |
| rs125701 | rs8191448 | rs16891982 |
| rs3810378 | rs8191446 | rs26722 |
| rs3213401 | rs8191445 | |
| rs3213274 | rs8191439 | |
| rs3213255 | rs8191438 | |
| rs3213245 | rs8191431 | |
| rs3213239 | rs6591256 | |

TABLE 2
RS numbers for SNPs associated with one or more areas of skin health

| rs number | Gene | Gene Name | Gene Function | Category | Notes | MAF | Chromosome |
|---|---|---|---|---|---|---|---|
| rs10934938 | COL29A1 | Collagen | epidermal | Collagen | Maintains the | 0.382 | 3q22.1 |
| | | type VI, | integrity and | | epidermal structure | | |
| | | alpha 5 | function | | and integrity of skin | | |
| rs1062470 | CDSN | Corneodes | Localizes to the | Collagen | Protein helps to | 0.4601 | 6p21.3 |
| | | mosin | epidermis and | | maintain proper | | |
| | | | undergoes a | | levels of skin cell | | |
| | | | series of | | production | | |
| | | | cleavages | | | | |
| | | | required for | | | | |
| | | | desquamation, | | | | |
| | | | involved in cell- | | | | |
| | | | cell adhesion in | | | | |
| | | | epidermis; | | | | |
| | | | linked to | | | | |
| | | | psoriasis | | | | |
| rs1107946 | COL1A1 | Collagen | Pro alpha 1 | Collagen | Maintains | 0.2525 | 17q21.33 |
| | | type I, | chain of type I | | underlying bone | | |
| | | alpha 1 | collagen, | | structure to keep | | |
| | | | abundant in | | skin from sagging | | |
| | | | bone, cornea, | | | | |
| | | | dermis, and | | | | |
| | | | tendon | | | | |
| rs2412298 | COL1A1 | Collagen | Pro alpha 1 | Collagen | Maintains | | 17q21.33 |
| | | type I, | chain of type I | | underlying bone | | |
| | | alpha 1 | collagen | | structure to keep | | |
| | | | | | skin from sagging | | |
| rs1799750 | MMP1 | Matrix | Breaks down | Collagen | Keeps collagen at | 0.4509 | 11q22.3 |
| | | metalloproteinase | collagen I, II, III, | | healthy levels | | |
| | | 1 | and VII | | | | |
| rs1042602 | TYR | Tyrosinase | Catalyzes | Sun | helps maintain | 0.1823/ | 11q14.3 |
| | | | conversion of | | healthy skin | 0.309 | |
| | | | tyrosine to | | pigmentation | | |
| | | | melanin | | | | |
| rs1408799 | TYRP1 | Tyrosinase- | part of melanin | Sun | helps maintain | 0.3499 | 9p23 |
| | | related | biosynthetic | | healthy skin | | |
| | | protein 1 | pathway; | | pigmentation | | |
| | | | increased | | | | |
| | | | melanoma risk | | | | |
| rs4911414 | ASIP | Agouti | paracrine | Sun | associated with an | 0.2305 | 20q11.2-q12 |
| | | Signaling | signaling | | increased | | |
| | | protein | molecule that | | melanoma risk | | |
| | | | causes hair | | | | |
| | | | follicle | | | | |
| | | | melanocytes to | | | | |
| | | | synthesize | | | | |
| | | | pheomelanin | | | | |
| rs1015362 | ASIP | Agouti | paracrine | Sun | associated with an | 0.3806 | 20q11.2-q12 |
| | | Signaling | signaling | | increased risk of | | |
| | | protein | molecule that | | non-melanoma skin | | |
| | | | causes hair | | cancers, cutaneous | | |
| | | | follicle | | malignant | | |
| | | | melanocytes to | | melanoma, and | | |
| | | | synthesize | | BCC | | |
| | | | pheomelanin | | | | |
| rs910873 | CDC91L1 | phosphatidy | attaches GPI- | Sun | look up snp in | 0.0216 | 20q11.22 |
| | | linositol | anchors to | | snpedia | | |
| | | glycan | proteins | | | | |
| | | anchor | | | | | |
| | | biosynthesis, | | | | | |
| | | class U | | | | | |
| rs258322 | MC1R | Melanocortin | Receptor for | Sun | Increased | 0.2677 | 16q24 |
| | | 1 receptor | melanocyte | | melanoma | | |
| | | | stimulating | | susceptibility | | |
| | | | hormone; within | | possibly due to | | |
| | | | CDK10 gene | | MC1R functional | | |
| | | | (cyclin | | variant | | |
| | | | dependent | | | | |
| | | | kinase 10 | | | | |
| | | | involved in cell | | | | |
| | | | cycle | | | | |
| | | | progression) | | | | |
| rs1805007 | MC1R | Melanocortin | Receptor for | Sun | increased risk of | 0.0303 | 16q24 |
| | | 1 receptor | melanocyte | | melanoma | | |
| | | | stimulating | | | | |
| | | | hormone; within | | | | |
| | | | CDK10 gene | | | | |
| | | | (cyclin | | | | |
| | | | dependent | | | | |
| | | | kinase 10 | | | | |
| | | | involved in cell | | | | |
| | | | cycle | | | | |
| | | | progression) | | | | |
| rs25487 | XRCC1 | X-ray repair | DNA base | Sun | increased risk of | 0.2631 | 19q13.2 |
| | | complementing | excision repair | | melanoma | | |
| | | defective | | | | | |
| | | repair in | | | | | |
| | | Chinese | | | | | |
| | | hamster | | | | | |
| | | cells 1 | | | | | |
| rs25489 | XRCC1 | X-ray repair | DNA base | Sun | increased risk of | 0.0606 | 19q13.2 |
| | | complementing | excision repair | | melanoma | | |
| | | defective | | | | | |
| | | repair in | | | | | |
| | | Chinese | | | | | |
| | | hamster | | | | | |
| | | cells 1 | | | | | |
| rs11052133 | hOGG1 | Oxoguanine | DNA damage | Sun | Increased risk of | 0.2985 | 3p26.2 |
| | | glycosylase | repair; removes | | T2DM in Mexican | | |
| | | 1 | mutagenic | | Americans; protein | | |
| | | | bases as a | | is downregulated in | | |
| | | | result of | | basal cell | | |
| | | | exposure to | | carcinoma; can be | | |
| | | | reactive oxygen | | under | | |
| | | | | | antioxidants/ | | |
| | | | | | inflammation too | | |
| rs910873 | CDCp1L1 | Phosphatidylinositol | Attaches GPI- | Sun | ASIP gene related, | 0.0216 | 20q11.22 |
| | | glycan | anchors to | | increased risk of | | |
| | | anchor | proteins. | | melanoma and | | |
| | | biosynthesis, | Involved in cell | | non-melanoma skin | | |
| | | class U | division control. | | cancer | | |
| rs4516035 | VDR | Vitamin D | Binds vitamin | Sun | increased risk of | 0.2149 | 12q13.11 |
| | | Receptor | D3 and | | malignant | | |
| | | | secondary bile | | melanoma | | |
| | | | acid lithocholic | | | | |
| | | | acid | | | | |
| rs11608363 | IL31 | Interleukin-31 | made | Inflammation | increased eczema | 0.483 | 12q24.31 |
| | | | principally by | | risk; may be | | |
| | | | activated Th2- | | involved in the | | |
| | | | type T cells, | | promotion of | | |
| | | | interacts with a | | allergic skin | | |
| | | | heterodimeric | | disorders and in | | |
| | | | receptor | | regulating other | | |
| | | | consisting of | | allergic diseases, | | |
| | | | IL31RA (MIM | | such as asthma | | |
| | | | 609510) and | | | | |
| | | | OSMR | | | | |
| rs8192284 | IL6R | Interleukin | Receptor for | Inflammation | elevated risk of | 0.3191 | 1q21 |
| | | 6 receptor | IL6 | | melanoma; | | |
| | | | | | increased risk for | | |
| | | | | | inflammation | | |
| rs6684439 | IL6R | Interleukin | Receptor for | Inflammation | elevated risk for | 0.3815 | 1q21 |
| | | 6 receptor | IL6 | | melanima | | |
| rs4845618 | IL6R | Interleukin | Receptor for | Inflammation | | 0.4761 | 1q21 |
| | | 6 receptor | IL6 | | | | |
| rs4845622 | IL6R | Interleukin | Receptor for | Inflammation | | 0.3196 | 1q21 |
| | | 6 receptor | IL6 | | | | |
| rs12093699 | CRP | C-reactive | Host defense, | Inflammation | | 0.2553 | 1q23.2 |
| | | protein | recognizes | | | | |
| | | | pathogens, | | | | |
| | | | inflammation | | | | |
| rs780094 | GCKR | Glucokinase | Inhibits | Inflammation | | 0.3857 | 2p23 |
| | | regulator | Glucokinase | | | | |
| rs7310409 | HNF1A | Hepatic | Trx factor for | Inflammation | | 0.3935 | 12q24.2 |
| | | nuclear | liver-specific | | | | |
| | | factor 1 | genes | | | | |
| | | alpha | | | | | |
| rs1892534 | LEPR | Leptin | stimulates gene | Inflammation | | 0.4509 | 1p31 |
| | | Receptor | trx through | | | | |
| | | | activation of | | | | |
| | | | cytosolic STAT | | | | |
| | | | proteins | | | | |
| rs17561 | IL1A | Interleukin | Involved in | Inflammation | Assoc with RA and | 0.2029 | 2q14 |
| | | 1A | inflammatory | | AD | | |
| | | | response | | | | |
| rs3093058 | CRP | C-reactive | Host defense, | Inflammation | Increased levels of | 0.0395 | 1q23.2 |
| | | protein | recognizes | | CRP | | |
| | | | pathogens, | | | | |
| | | | inflammation | | | | |
| rs3091244 | CRP | C-reactive | Host defense, | Inflammation | Increased levels of | 0.2608 | 1q23.2 |
| | | protein | recognizes | | CRP | | |
| | | | pathogens, | | | | |
| | | | inflammation | | | | |
| rs3093062 | CRP | C-reactive | Host defense, | Inflammation | Increased levels of | 0.0395 | 1q23.2 |
| | | protein | recognizes | | CRP | | |
| | | | pathogens, | | | | |
| | | | | inflammation | | | |
| rs180047 | CRP | C-reactive | Host defense, | Inflammation | Increased levels of | 0.247 | 1q23.2 |
| | | protein | recognizes | | CRP | | |
| | | | pathogens, | | | | |
| | | | inflammation | | | | |
| rs3093066 | CRP | C-reactive | Host defense, | Inflammation | Increased levels of | 0.068 | 1q23.2 |
| | | protein | recognizes | | CRP | | |
| | | | pathogens, | | | | |
| | | | inflammation | | | | |
| rs2808630 | CRP | C-reactive | Host defense, | Inflammation | Increased levels of | 0.2149 | 1q23.2 |
| | | protein | recognizes | | CRP | | |
| | | | pathogens, | | | | |
| | | | inflammation | | | | |
| rs12093699 | CRP | C-reactive | Host defense, | Inflammation | near the gene | 0.2553 | 1q23.2 |
| | | protein | recognizes | | | | |
| | | | pathogens, | | | | |
| | | | inflammation | | | | |
| rs1205 | CRP | C-reactive | Host defense, | Inflammation | | 0.365 | 1q23.2 |
| | | protein | recognized | | | | |
| | | | pathogens, | | | | |
| | | | inflammation | | | | |
| rs2250417 | IL8 | Interleukin | | Inflammation | T2DM risk | 0.3407 | 4q13-q21 |
| | | 8 | | | | | |
| rs8192284 | IL6R | Interleukin | Receptor for | Inflammation | T2DM risk | 0.3191 | 1q21 |
| | | 6 receptor | IL6 | | | | |
| rs1001179 | CAT | Catalase | Converts H2O2 | Antioxidants | | 0.123 | 11p13 |
| | | | to oxygen and | | | | |
| | | | water | | | | |
| rs769214 | CAT | Catalase | Converts H2O2 | Antioxidants | | 0.4775 | 11p13 |
| | | | to oxygen and | | | | |
| | | | water | | | | |
| rs2300181 | CAT | Catalase | Converts H2O2 | Antioxidants | | 0.2172 | 11p13 |
| | | | to oxygen and | | | | |
| | | | water | | | | |
| rs7943316 | CAT | Catalase | Converts H2O2 | Antioxidants | increased H2O2 | 0.4894 | 11p13 |
| | | | to oxygen and | | induced DNA | | |
| | | | water | | damage | | |
| rs1050450 | GPX1 | Glutathione | Detoxifies | Antioxidants | | 0.2493 | 3p21.3 |
| | | peroxidase | H2O2 to | | | | |
| | | 1 | oxygen and | | | | |
| | | | water | | | | |
| rs1800668 | GPX1 | Glutathione | Detoxifies | Antioxidants | | 0.2346 | 3p21.3 |
| | | peroxidase | H2O2 to | | | | |
| | | 1 | oxygen and | | | | |
| | | | water | | | | |
| rs1799983 | NOS3 | Nitric oxide | Forms NO | Antioxidants | | 0.197 | 7q36 |
| | | synthase 3 | during conv. Of | | | | |
| | | | arginine to | | | | |
| | | | citrulline | | | | |
| rs662 | PON1 | Paraoxonase | Binds to HDL | Antioxidants | | 0.4835 | 7q21.3 |
| | | 1 | and detoxes | | | | |
| | | | organophospha | | | | |
| | | | tes | | | | |
| rs2070424 | SOD1 | Superoxide | Catalyzes | Antioxidants | | 0.2388 | 21q22.11 |
| | | Dismutase | conversion SO | | | | |
| | | 1 | radical to H2O2 | | | | |
| rs2234694 | SOD1 | Superoxide | Catalyzes | Antioxidants | adjacent to splice | 0.023 | 21q22.11 |
| | | Dismutase | conversion SO | | site boundary | | |
| | | 1 | radical to H2O2 | | | | |
| rs4880 | SOD2 | Superoxide | Catalyzes | Antioxidants | alters Mn SOD2 | 0.3705 | 6q25.3 |
| | | Dismutase | conversion SO | | expression | | |
| | | 2 | radical to H2O2 | | | | |
| rs699473 | SOD3 | Superoxide | Catalyzes | Antioxidants | | 0.45 | 4p15.2 |
| | | Dismutase | conversion SO | | | | |
| | | 3 | radical to H2O2 | | | | |
| rs1800562 | HFE | Hemochro | Regulates iron | Antioxidants | HFEC282Y | 0.0197 | 6p21.3 |
| | | matosis | absorption by | | | | |
| | | | regulating the | | | | |
| | | | interaction of | | | | |
| | | | the transferrin | | | | |
| | | | receptor with | | | | |
| | | | transferrin | | | | |
| rs2266637 | GSTT1 | Glutathione | Catalyzes | Antioxidants | | 0.0468 | 22q11.23 |
| | | S | conjugation of | | | | |
| | | transferase | reduced | | | | |
| | | theta 1 | glutathione to | | | | |
| | | | electrophilic | | | | |
| | | | and | | | | |
| | | | hydrophobic | | | | |
| | | | compounds | | | | |
| rs1695 | GSTP1 | Glutathione | Catalyzes | Antioxidants | increased risk if | 0.3246 | 11q13 |
| | | S | conjugation of | | asthma | | |
| | | transferase | hydrophobic | | | | |
| | | pi 1 | and | | | | |
| | | | electrophilic | | | | |
| | | | compounds | | | | |
| | | | with reduced | | | | |
| | | | glutathione | | | | |
| rs1138272 | GSTP1 | Glutathione | Catalyzes | Antioxidants | increased risk if | 0.0354 | 11q13 |
| | | S | conjugation of | | asthma | | |
| | | transferase | hydrophobic | | | | |
| | | pi 1 | and | | | | |
| | | | electrophilic | | | | |
| | | | compounds | | | | |
| | | | with reduced | | | | |
| | | | glutathione | | | | |
| rs4880 | MnSOD | Manganese | Converts | Antioxidants | increased oxidative | 0.3705 | 6q25.3 |
| | | superoxide | superoxide | | stress | | |
| | | dismutase | compounds to | | | | |
| | | | H2O2 and | | | | |
| | | | water | | | | |
| rs11568821 | PDCD1 | Programmed | Cell surface | Antioxidants | G>A at 4th intron | 0.482 | 2q37.3 |
| | | cell death | receptor | | affects RUNX1 | | |
| | | 1 | involved in | | binding; | | |
| | | | B-cell | | methylation site | | |
| | | | differentiation | | | | |
| rs1800470 | TGFB1 | Transforming | Regulates | Antioxidants | codon 10-869 T/C | | |
| | | growth | proliferation, | | TC and TT (high); | | |
| | | factor beta | differentiation, | | CC (intermed.) TC | | |
| | | 1 | adhesion, | | and CC correlated | | |
| | | | migration, and | | with early onset, TT | | |
| | | | other functions | | with late onset | | |
| | | | in many cell | | | | |
| | | | types | | | | |
| rs1800471 | TGFB1 | Transforming | Regulates | Antioxidants | codon 25-915 G/C | 0.444 | 19q13.1 |
| | | growth | proliferation, | | GG common | | |
| | | factor beta | differentiation, | | | | |
| | | 1 | adhesion, | | | | |
| | | | migration and | | | | |
| | | | other functions | | | | |
| | | | in many cell | | | | |
| | | | types | | | | |
| rs4911414 | TNFa | Tumor | Secreted by | Antioxidants | MAF~.09- ow | 0.2305 | 6p21.3 |
| | | necrosis | macrophages | | | | |
| | | factor alpha | and involved in | | | | |
| | | | cell | | | | |
| | | | proliferation, | | | | |
| | | | differentiation, | | | | |
| | | | apoptosis, lipid | | | | |
| | | | metabolism, | | | | |
| | | | and coagulation | | | | |
| rs2917666 | NQO1 | NADPH | Reduces | Antioxidants | Assoc. between | 0.4949 | 16q22.1 |
| | | dehydrogenase | quinones to | | NO2 and asthma | | |
| | | quinone 1 | hydroquinones— | | prevalence | | |
| | | | COQ10 | | | | |
| | | | reductase | | | | |
| rs2070600 | AGER/ | Advanced | Interacts with | Glycation | impacts levels of | 0.07438 | 6p21.3 |
| | RAGE | Glycation | AGEs and | | circulating AGEs, | | |
| | | End | other molecules | | they form at an | | |
| | | Product- | implicated in | | accelerated rate in | | |
| | | specific | homeostasis, | | diabetes | | |
| | | Receptor | development, | | | | |
| | | | and | | | | |
| | | | inflammation | | | | |
| rs11052133 | hOGG1 | Oxoguanine | DNA damage | Glycation | Increased risk of | 0.2985 | 3p26.2 |
| | | glycosylase | repair | | T2DM in Mexican | | |
| | | 1 | | | Americans | | |
| rs1137101 | LEPR | Leptin | Binds leptin; | Glycation | Helps maintain | 0.4104 | 1p31 |
| | | Receptor | involved in | | healthy levels of | | |
| | | | glucose | | blood sugar | | |
| | | | homeostasis | | | | |

1. A method of characterizing a subject's skin, comprising generating a personalized skin profile by generating a personalized DNA report in which the personalized DNA report comprises an overall risk ratings for the likelihood of acquiring a deficiency in one or more areas of skin health and/or beauty, thereby revealing the subject's genetic strengths, weaknesses and/or risks related to the one or more areas of skin health and/or beauty thereby allowing a personalized skincare and/or nutritional regimen to be developed and implemented.

2. The method of claim 1, wherein generating a personalized DNA report comprises electronically creating the DNA report through use a computer system.

3. The method of claim 1, wherein the overall risk ratings for the likelihood of acquiring a deficiency in skin health and/or beauty comprises a rating system of a high, a medium and/or a low risk rating for acquiring a deficiency in one or more areas of skin health and/or beauty.

4. The method of claim 3, wherein the rating system comprises assigning a value of 0.25 to a result of homozygous for the non-ideal allele at an at risk single nucleotide polymorphism (SNP) location (non-ideal function); assigning a value of 0.50 to a result of heterozygous for the non-ideal allele at the risk SNP location; and assigning a value of 1.00 to a result of homozygous for the ideal allele at the risk SNP location; whereby high risk is considered an average numerical value of 0.25-0.5; medium risk is considered to be a numerical average of 0.5-0.75; and low risk is considered to be a numerical value of 0.75-1.0.

5. The method of claim 4, wherein the DNA report further comprises a description of one or more biological effects and/or visible signs of the one or more areas of skin health and/or beauty.

6. The method of claim 5, wherein the one or more areas of skin health comprise collagen formation, sun protection, antioxidant protection, glycation protection or inflammation control.

7. The method of claim 6, wherein the one or more areas of skin health consists collagen formation properties, sun protection properties, antioxidant protection properties, glycation protection properties and inflammation control properties.

8. The method of claim 7, wherein generating a personalized DNA report comprises determining a subject's genetic potential in the one or more areas of skin health and/or beauty by analyzing one or more skin health-associated genetic markers, such as one or more single nucleotide polymorphisms (SNPs) associated with the one or more areas of skin health and/or beauty being assessed.

9. The method of claim 8, wherein the one or more skin health-associated molecular markers is a single nucleotide polymorphism (SNP) associated with the one or more areas of skin health and/or beauty, such as one or more SNPs with RS numbers provided in Table 1 and/or 2.

10. The method of claim 1, further comprising obtaining a biological sample from the subject prior to generating the personalized DNA report.

11. The method of claim 1, further comprising identifying one or more genetic markers, such as one or more SNPs, associated with a particular area of skin health and/or beauty.

12. The method of claim 1, further comprising providing the generated personalized skin profile to the subject and recommending and/or providing one or more skincare treatments and/or nutritional regimens to the subject based upon the personalized skin profile generated by the characterization analysis.

13. The method of claim 12, wherein recommending and/or providing one or more skincare and/or nutritional regimens comprises recommending one or more non-customized skincare products and one or more customized skincare products.

14. The method of claim 13, wherein the one or more non-customized skincare products comprises an antioxidant cleanser, a balancing toner and/or an antioxidant moisturizer.

15. The method of claim 13, wherein the one or more customized skincare products comprise a combination of one or more treatment serums and one or more nutritional products based upon the overall personalized DNA report.

16. The method of claim 15, wherein the one of more treatment serums comprises vitamin C treatment serum, hyaluronic moisture treatment serum, wrinkle treatment serum, calming treatment serum or a combination thereof.

17. The method of claim 15, wherein the one or more nutritional supplements comprises an antioxidant defense supplement, glycation defense supplement, a sun defense supplement, a collagen defense supplement, an inflammation defense supplement or any combination thereof.
